(19) United States
(12) Patent Application Publication
(10) Pub. No.: US 2023/0265128 A1
(43) Pub. Date: Aug. 24, 2023

(54) STABILISED VIRAL FUSION PROTEINS

(71) Applicant: Oxford University Innovation Limited, Oxford (Oxfordshire) (GB)

(72) Inventors: Alexander DOUGLAS, Oxford (Oxfordshire) (GB); Sofiya FEDOSYUK, Oxford (Oxfordshire) (GB)

(21) Appl. No.: 17/910,598

(22) PCT Filed: Mar. 12, 2021
(86) PCT No.: PCT/GB2021/050636

§ 371 Date: Sep. 9, 2022

(30) Foreign Application Priority Data
Mar. 12, 2020 (GB) 2003611.7

(51) Int. Cl. C07K 14/005; A61K 39/12; C12N 15/86

(52) U.S. Cl. CPC C07K 14/005; A61K 39/12; C12N 15/86; A61K 38/00

(57) ABSTRACT

The invention relates to stabilised pre-fusion conformation Class III fusion proteins. The inve ntion also provides vaccine compositions for immunising a subject against viral infections.

### FIELD OF THE INVENTION

The invention relates to stabilised pre-fusion conformation Class III fusion proteins. The invention also provides vaccine compositions for immunising a subject against viral infections.

### BACKGROUND OF THE INVENTION

To enter host cells, enveloped viruses require fusion of the viral envelope with the host cell membrane. Fusion of the viral envelope with the host cell membrane is mediated by fusion proteins within the viral envelope. These viral fusion proteins are conformationally-metastable proteins which, upon triggering by some factor signifying contact with a host cell, transition from a higher-energy pre-fusion conformation to a lower-energy post-fusion conformation, via an unstable intermediate state. The intermediate state exposes hydrophobic fusion peptides or fusion loops which insert into the host cell membrane; the transition to the lower-energy post-fusion conformation pulls the two membranes together, driving fusion.

The post-fusion conformation of viral fusion proteins is typically the lower-energy state. Consequently, attempts to produce viral fusion proteins recombinantly often favour the production of viral fusion proteins in the post-fusion conformation, especially when a viral fusion protein is expressed without the context of the cell membrane. However, the epitopes which stimulate the production of neutralising antibodies, are often contained within the pre-fusion conformation, and in some cases are absent in the post-fusion form. Accordingly, immunisation using a protein in pre-fusion conformation is clinically desirable as it has the potential to induce substantially more potent neutralising antibody than immunisation with post-fusion conformation protein. More broadly, it is recognised that means of stabilising fusion proteins in the authentic conformation found on the surface of the virion can improve the protein’s efficacy as a vaccine. Viral fusion proteins have been classified into Class I (including the fusion proteins of human immunodeficiency virus and respiratory syncytial virus), Class II (including the fusion proteins of dengue virus) and Class III (including the rhabdoviruses and herpesviruses). Class III proteins form trimeric spikes on the viral envelopes, which have a squat tripod-like pre-fusion conformation and are elongated in the post-fusion conformation. Although Class III fusion proteins share some similarities with Class I fusion proteins, notably the presence in the post-fusion form of a central bundle of three alpha-helices, they differ from Class I fusion proteins in a number of important respects. One key difference is the tripod-like structure of the pre-fusion Class III conformation, in which the fusion loops, and probably also the transmembrane domains passing through the viral envelope, are substantially separated from each other. In contrast, the transmembrane domains of Class I fusion proteins are closely associated. A further difference between the Class I and Class III fusion proteins is that those in Class III generally do not require proteolytic cleavage for activation, whereas those in Class I do. The architecture and connectivity of the various domains of the Class I and Class III fusion proteins are also quite different. For example, the domains carrying Class III proteins’ fusion loops have similarities to those of Class II proteins but are completely different from those of Class I.

Stabilisation of several Class I proteins in pre-fusion conformation has been reported. It is notable that a small number of techniques have proven effective across relatively distantly related viruses with Class I fusion proteins. Such techniques include forced trimerisation at the C-terminus, manipulation of the proteolytic cleavage sites, and certain point mutations targeted to interfere with secondary structure transitions or to introduce artificial disulphide bonds. However, because of the structural differences between Class I, II and III fusion proteins, the strategies for stabilising Class I or Class II proteins cannot be directly extrapolated to Class III protein. Therefore, to-date only one pre-fusion structure of a Class III fusion protein has been described (Vesicular Stomatitis Virus (VSV) protein G - VSVG), with an additional structure published of a rabies virus glycoprotein (RVG) monomer at pH 8.0 (which probably has some similarity to the pre-fusion structure but also has important differences, including a likely non-natural arrangement of the C-terminal region and lack of trimeric organisation). There are no published reports of the successful design of stabilised pre-fusion configurations of any Class III fusion proteins in a format suitable for vaccination. Although there are reports of mutants with loss of fusion function, it has not to our knowledge been demonstrated that any of these mutations stabilise a recombinant antigen in pre-fusion conformation, nor that any of them suggest a strategy with potential general applicability across the Class III proteins.

Class III proteins are expressed on several clinically significant viruses, and so potentially represent important vaccine immunogens. Rabies virus glycoprotein (RVG) is the target of neutralising antibodies which are known to provide a protective effect. Some of these antibodies, in particular those against antigenic site II of RVG, are known to bind only under neutral pH conditions (when the protein is in the pre-fusion conformation), and lose binding under acidic pH conditions (when the protein adopts the post-fusion conformation). The Class III fusion proteins of the herpesvirus family (known as glycoprotein B) are also known to be targets of neutralising antibodies. Clinical trials of vaccines based upon human cytomegalovirus gB have demonstrated partial efficacy. There are also indications that non-neutralising gB-binding antibodies may have some protective effect.

Thus, there is a need for new and improved vaccines and protective antibodies against human and veterinary pathogens with Class III fusion proteins. In particular, there is a need for stabilised forms of the pre-fusion conformation of Class III fusion proteins which can be recombinantly expressed, displaying stable pre-fusion conformation epitopes capable of stimulating the production of neutralising antibodies. Subunit vaccines based upon stabilised Class III proteins in the pre-fusion conformation have the potential to provide improved efficacy over existing vaccines or post-fusion conformations of the proteins. In addition, the use of stabilised Class III proteins in the pre-fusion conformation provide other advantages, including manufacturing advantages such as lower cost than vaccines based upon whole viruses.

The project leading to this application has received funding from the European Union’s Horizon 2020 research and innovation programme under the Marie Sklodowska-Curie grant agreement No 840866.

### SUMMARY OF THE INVENTION

The present invention provides stabilised pre-fusion Class III fusion protein and immunogenic fragment thereof, together with products for use in methods of vaccinating a subject.

The present inventors have identified a region, common to all Class III fusion proteins, that when mutated prevents the transition of the Class III fusion protein from the pre-fusion conformation to the post-fusion conformation. The stabilised pre-fusion conformation Class III fusion proteins retain and display the pre-fusion conformation specific epitopes found on the corresponding wild-type (WT) protein. Thus, the proteins developed by the present inventors can be used as antigens to stimulate the production of pre-fusion conformation specific neutralising antibodies. In addition, the present inventors have successfully expressed recombinant stabilised proteins according to the invention.

Accordingly, the invention provides a stable pre-fusion Class III fusion protein, or an immunogenic fragment thereof, comprising one or more mutations that prevent formation of the central extended helix in the post-fusion conformation trimerisation interface.

In some embodiments, said one or more mutation that prevents the formation of the central extended helix is a mutation that prevents extension of the pre-fusion conformation central helix into the post-fusion central extended helix.

In some embodiments, said central extended helix in the post-fusion conformation trimerisation interface corresponds to, or aligns with:
(a) the extended helix C in rabies virus glycoprotein (RVG);
(b) helix F of the trimerisation domain of vesicular stomatitis virus glycoprotein (VSVG); and/or
(c) helix alpha-C of the trimerisation domain of Epstein-Barr virus glycoprotein B (EBV gB).

In some embodiments, said central extended helix in the post-fusion conformation trimerisation interface is defined as extending, at its N-terminal end, up to the 32nd amino acid residue N-terminal to the conserved cysteine residue of the Class III fusion protein and, at its C-terminal end, up to the 16th amino acid residue C-terminal to the conserved cysteine of the Class III fusion protein; wherein optionally the conserved cysteine residue corresponds to, or aligns with:
(a) amino acid residue 283 of the RVG sequence according to SEQ ID NO: 3;
(b) amino acid residue 284 of the VSVG sequence according to SEQ ID NO: 5;
(c) amino acid residue 484 of the EBV gB sequence according to SEQ ID NO: 6;
(d) amino acid residue 507 of the cytomegalovirus glycoprotein B (CMV gB) sequence according to SEQ ID NO: 8; and/or
(e) amino acid residue 529 of the herpes simplex virus glycoprotein B (HSV-1 gB) sequence according to SEQ ID NO: 10.
(f) amino acid residue 526 of the herpes simplex virus glycoprotein B (HSV-2 gB) sequence according to SEQ ID NO: 32.

In some embodiments, said central extended helix of the post-fusion conformation trimerisation interface corresponds to, or aligns with amino acid residues:
(a) 262 to 293 of the RVG sequence of SEQ ID NO: 3;
(b) 263 to 294 of the VSVG sequence of SEQ ID NO: 5;
(c) 458 to 500 of the EBV gB sequence of SEQ ID NO: 6;
(d) 481 to 520 of the CMV gB sequence of SEQ ID NO: 8; and/or (e) 503 to 545 of the HSV-1 gB sequence of SEQ ID NO: 10.

In some embodiments, the Class III fusion protein is a: (a) RVG; (b) VSVG; or (c) herpesvirus glycoprotein B, wherein optionally said herpesvirus is selected from a cytomegalovirus, Epstein- Barr virus, herpes simplex virus-1 and/or herpes simplex virus-2.

In some embodiments, said one or more mutation is an amino acid substitution.

In some embodiments, said amino acid substitution is a non-conservative amino acid substitution.

In some embodiments, said one or more mutation is:
(a) a helix-disrupting mutation and said amino acid substitution is a substitution by proline; and/or
(b) an amino acid substitution by an amino acid with increased hydrophobicity compared with the amino acid being substituted, wherein said amino acid substitution is independently selected from a substitution by leucine, alanine, isoleucine or valine.

In some embodiments, said one or more mutation is at an amino acid corresponding to, or aligning with, position 270, 271, 272 and/or 273 of the RVG sequence of SEQ ID NO: 3.

In some embodiments, the amino acid corresponding to position:
(a) 270 of the RVG sequence of SEQ ID NO: 3 is substituted by proline;
(b) 271 of the RVG sequence of SEQ ID NO: 3 is substituted by proline;
(c) 272 of the RVG sequence of SEQ ID NO: 3 is substituted by proline; and/or
(d) 273 of the RVG sequence of SEQ ID NO: 3 is substituted by proline.

In some embodiments, the Class III fusion protein is a RVG and wherein said protein comprises one or more amino acid substitutions selected from H270P, L271P, V272P and/or V273P.

In some embodiments, the protein or immunogenic fragment thereof is a RVG comprising an amino acid sequence selected from SEQ ID NOs: 11, 12, 13 or 14.

In some embodiments, the protein or immunogenic fragment thereof of comprises one or more mutation is at an amino acid corresponding to, or aligning with, position 516, 517, 518 and/or 519 of the HSV gB sequence of SEQ ID NO: 10. In some embodiments, the amino acid corresponding to position:
(a) 516 of the HSV gB sequence of SEQ ID NO: 10 is substituted by proline;
(b) 517 of the HSV gB sequence of SEQ ID NO: 10 is substituted by proline;
(c) 518 of the HSV gB sequence of SEQ ID NO: 10 is substituted by proline; and/or
(d) 519 of the HSV gB sequence of SEQ ID NO: 10 is substituted by proline.

In some embodiments, the Class III fusion protein is an HSV gB and wherein said protein comprises one or more amino acid substitutions selected from H516P, V517P, N518P and/or D519P.

In some embodiments, the protein or immunogenic fragment thereof is an HSV gB comprising an amino acid sequence selected from SEQ ID NOs: 27, 28, 29 or 30.

The invention also provides a stable pre-fusion Class III fusion protein, or an immunogenic fragment thereof, comprising: (a) one or more non-conservative amino acid substitutions within its pre-fusion conformation central helix; and/or (b) one or more non-conservative amino acid substitutions within amino acid residues corresponding to or aligning with amino acid residues: (i) 262 to 293 of the RVG sequence of SEQ ID NO: 3; (ii) 263 to 294 of the VSVG sequence of SEQ ID NO: 5; (iii) 458 to 500 of the EBV gB sequence of SEQ ID NO: 6; (iv) 481 to 520 of the CMV gB sequence of SEQ ID NO: 8; and/or (v) 503 to 545 of the HSV-1 gB sequence of SEQ ID NO: 10; wherein preferably the one or more non-conservative amino acid substitution is substitution by proline.

In some embodiments, the protein or immunogenic fragment thereof of the invention further comprises one or more additional mutations in a region other than the central extended helix of the post-fusion conformation trimerisation interface.

In some embodiments, said one more additional mutation is a mutation in an amino acid corresponding to or aligning with position:
(a) 261 of the RVG sequence of SEQ ID NO: 3;
(b) 262 of the VSVG sequence of SEQ ID NO: 5;
(c) 462 of the EBV gB sequence of SEQ ID NO: 6;
(d) 485 of the CMV gB sequence of SEQ ID NO: 8; and/or
(e) 507 of the HSV gB sequence of SEQ ID NO: 10;
wherein optionally said amino acid mutation is an amino acid substitution by an amino acid with increased hydrophobicity compared to the amino acid being substituted.

In some embodiments, there is provided a protein or immunogenic fragment thereof of the invention, which induces neutralising antibodies against one or more epitope of the pre-fusion Class III fusion protein. The protein or immunogenic fragment thereof may induce neutralising antibodies against (i) antigenic site I; (ii) antigenic site II; and/or (iii) antigenic site III.

The invention further provides a protein or immunogenic fragment thereof of the invention for use in a vaccine.

The invention further provides a polynucleotide molecule encoding a protein of the invention

The invention further provides a viral vector, DNA vector and/or RNA vector:
(a) comprising a polynucleotide of the invention; and/or
(b) encoding a protein or immunogenic fragment thereof of the invention.

The invention further provides a virus-like particle, comprising a protein of the invention.

The invention further provides a vaccine composition, comprising a protein according to the invention, a polynucleotide molecule according to the invention, a viral vector and/or DNA vector and/or RNA vector according to the invention, and/or a virus-like particle according to the invention, and optionally a pharmaceutically acceptable excipient.

The invention further provides an antibody, or binding fragment thereof, that specifically binds to the protein or immunogenic fragment thereof of the invention.

The invention further provides a protein according to the invention, and/or a vaccine composition according to the invention, and/or a polynucleotide according to the invention, and/or a viral vector and/or DNA vector and/or RNA vector according to the invention and/or a virus-like particle according to the invention and/or a antibody of according to the invention, for use in a method of immunising a subject against a viral infection.

The invention further provides use of a protein according to the invention, and/or a vaccine composition according to the invention, and/or a polynucleotide according to the invention, and/or a viral vector and/or DNA vector and/or RNA vector according to the invention, and/or a virus-like particle according to the invention, and/or a antibody according to the invention, in the manufacture of a medicament for the immunisation of a subject against a viral infection.

In some embodiments, a protein, vaccine composition, polynucleotide, viral vector, DNA vector, RNA vector, virus-like particle and/or antibody for use according to the invention; or the use of a protein, vaccine composition, polynucleotide, viral vector, DNA vector, RNA vector, virus-like particle and/or antibody according to the invention; wherein said subject is a mammalian subject, preferably a human subject.

In some embodiments there is provided a protein, vaccine composition, polynucleotide, viral vector, DNA vector, RNA vector, virus-like particle and/or antibody for use according to the invention; or the use of a protein, vaccine composition, polynucleotide, viral vector, DNA vector, RNA vector, virus-like particle and/or antibody according to the invention; wherein said viral infection is a rhabdovirus infection or a herpesvirus infection..

The invention further provides use of a protein according to the invention for the generation of an antibody, or binding fragment thereof, specific to an epitope of a pre-fusion conformation Class III fusion protein.

### BRIEF DESCRIPTION OF THE DRAWINGS

FIG. 1: Alignment of the primary amino acid sequences of the central extended helix region in the post-fusion conformation trimerisation interface of VSVG, RVG, EBV gB, CMV gB and HSV-1 gB.

FIG. 2: Crystal structure of both the pre-fusion and post-fusion conformations of (A) the full length and (B) the core region of RVG

FIG. 3: Identification of the mutant forms of RVG with increased pre-fusion stability using FACS analysis. Membrane-bound RVG protein variants were labelled with primary pre-fusion-specific antibodies (1112-1 mAb) and subsequent fluorophore-conjugated secondary antibodies (anti-mouse APC). X-axis shows the total expression level of the pre-fusion protein expressed as median fluorescent signal (MFI) of antibody staining at pH 7.4. Y-axis shows protein stability as a ratio of MFI of antibody staining at pH 5.8 over MFI of antibody staining at pH 7.4. Each data point is an average between two independent biological replicates with both individual values indicated as an error bar.

FIG. 4: Model of the structure of the core region of EVB gB in pre-fusion conformation.

FIG. 5: Coomassie Blue stained SDS-PAGE gel of stabilised and purified EBV gB and CMV gB proteins showing purified mutant proteins and WT proteins, all of which were obtained at yields in excess of 10 micrograms/mL of culture supernatant. The presence of two fragments of EBV gB reflects the expected proteolytic processing. In the CMV gB construct, the furin cleavage site is mutated, resulting in a single predominant band.

FIG. 6: Size-exclusion profiles of the indicated class III fusion proteins.

FIG. 7: (A) RVG H270P is recognised by mAbs against antigenic site I, II and III. (B) RVG H270P pre-fusion conformation stabilisation is evident with antigenic site I-specific mAb, RVC20.

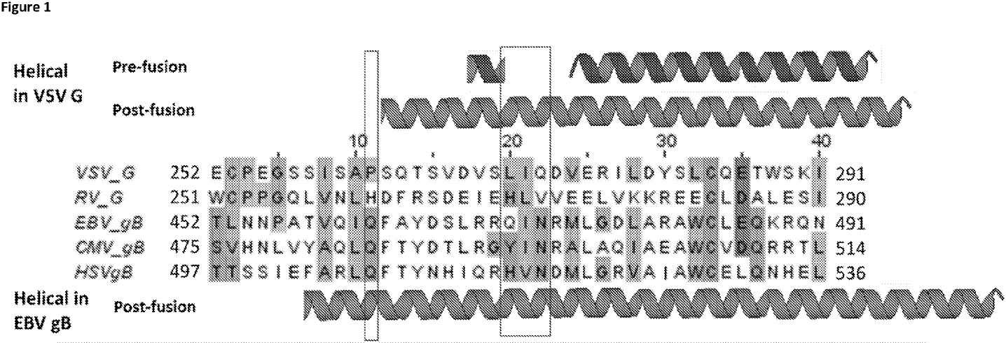

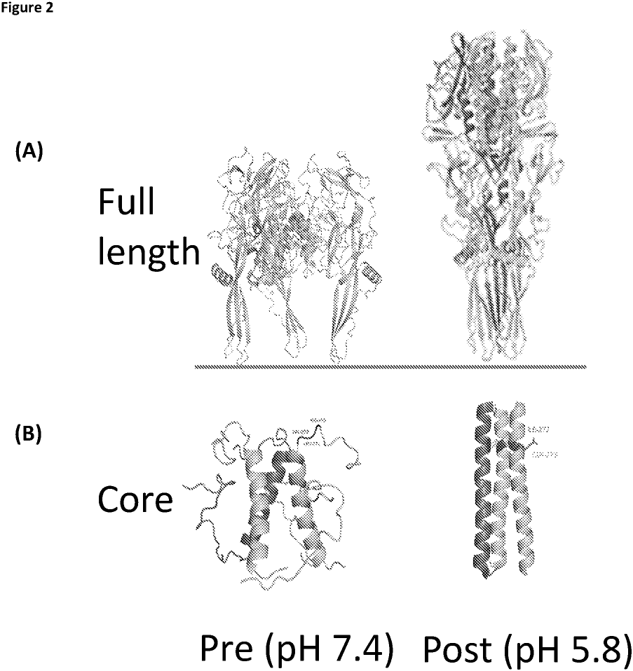

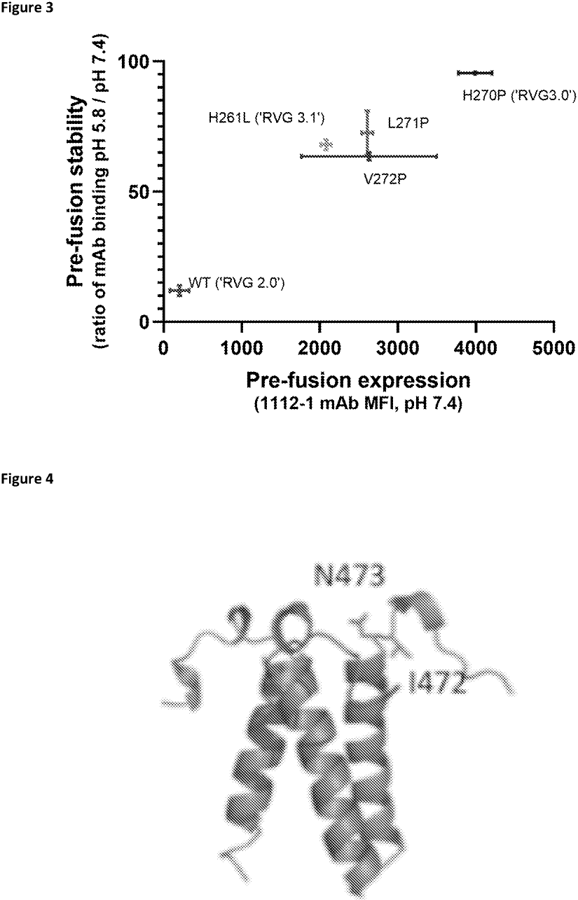

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The publications discussed herein are provided solely for their disclosure prior to the filing date of the present application. Nothing herein is to be construed as an admission that such publications constitute prior art to the claims appended hereto.

### Stabilised Pre-Fusion Class III Fusion Proteins

Class III fusion proteins share a common structural organisation with four to five domains. In the terminology of Roche et al. Science 315, 843-848 (2007), Domain IV is a fusion module which forms an extended β sheet and contains the fusion loops. The fusion loop is formed of a single continuous length of the primary sequence, embedded between two β-strands of domain III, which has a pleckstrin-homology like (PH) fold. Domain III, in turn, is contained within a largely helical domain II, which forms the trimerisation core of the protein. Domain II, in turn, is embedded within domain I, which is an external lateral or crown domain, according to the protein. Beyond the C-terminal end of domain I is a further helical region of the trimerisation domain (III), followed by the C-terminal region extending to the transmembrane domain. In the case of the herpesvirus gB proteins, this C-terminal region includes an additional fifth domain (domain V), lying on the surface of the protein in the reported post-fusion structures. Numbering of these domains varies e.g. Heldwein et al. Science 313, 217-220 (2006) use the reverse numerical ordering (with domain I being fusion, through to domain IV being the crown), but the overall architecture of the domains and their position relative to each other both in the primary sequence and in the folded proteins is conserved. As discussed above it has been shown that the proteins adopt a tripod-like pre-fusion conformation, with the ‘legs’, based on the virion envelope, composed of the fusion domains and probably the C-termini as they approach the transmembrane domain.

Crystallographic structures of several post-fusion conformation Class III fusion proteins have been reported (e.g. EBV gB - Backovic et al. Proc Natl Acad Sci U S A 106, 2880-2885 (2009), CMV gB Chandramouli et al. Nat Commun 6, 8176 (2015) and HSV-1 gB - Heldwein et al. Science 313, 217-220 (2006)). However, to date a single pre-fusion conformation Class III fusion protein, VSV G, has been reported in trimeric form. In addition, a structure of a monomeric form of RVG (Yang et al. Cell Host & Microbe 27, 1-13 (2020)) has been reported which is pre-fusion-like but the authors acknowledge includes a placement of the C-terminus which suggests it is in fact a non-natural conformation or intermediate form.

There are reports in the literature of mutant forms of some of these proteins which may affect their ability to mediate fusion, or in some cases their sensitivity to pH. There has however been no demonstration that such mutants result in the production of a form of the protein which is both expressed and effectively stabilised in such a way as to be potentially useful as a vaccine.

The inventors have for the first time demonstrated that a conserved region within the Class III fusion proteins, which forms part of a central extended helix in the post-fusion conformation, can be mutated to produce stabilised pre-fusion conformations of Class III fusions proteins.

Accordingly, the invention provides a stable pre-fusion Class III fusion protein, or an immunogenic fragment thereof, comprising one or more mutations that prevent formation of the central extended helix in the post-fusion conformation trimerisation interface. Said one or more mutation that prevent the formation of the central extended helix in the post-fusion conformation trimerisation interface is typically found within the central extended helix in the post-fusion conformation trimerisation interface. Without being bound by theory, it is believed that mutations in this region prevent the formation of the central extended helix, for example by preventing the extension of the shorter central helix that is present in the pre-fusion conformation into the central helix of the post-fusion conformation. Importantly, mutations in this region do not disrupt the shorter central helix of the pre-fusion conformation and, therefore, expression of the pre-fusion conformation is possible.

Thus, typically said one or more mutation that prevents the formation of the central extended helix is a mutation that prevents extension of the pre-fusion conformation central helix into the post-fusion central extended helix, without disrupting the pre-fusion conformation central helix. Preferably said one or more mutation that prevents the formation of the central extended helix does not disrupt expression of the stabilised pre-fusion conformation Class III protein.

The expression “stable pre-fusion Class III fusion protein” is intended to refer to a mutated Class III fusion protein, the pre-fusion conformation of which exhibits increased stability when compared to the pre-fusion conformation of a non-mutated (or wild-type (WT)) Class III fusion protein. Typically, the pre-fusion conformation of the mutated Class III fusion protein of the invention will exhibit increased stability when compared to the pre-fusion conformation of the non-mutated Class III fusion protein from which it is derived. Methods of determining the stability of the pre-fusion conformation of Class III fusion proteins are well known in the art. By way of non-limiting example, the stability of the pre-fusion conformation of some Class III fusion proteins may be determined by incubating a Class III fusion protein with a pre-conformation specific antibody at both neutral and acidic pH and determining antibody binding by ELISA or FACS (for example, the 1112-1 mAb and RVC20 mAb). As an alternative example, the stability of a pre-fusion conformation of a Class III fusion protein may be determined by exposing said protein to conditions under which at least a portion of the wild-type protein would be expected to adopt the post-fusion conformation, and assaying the proportion of protein retaining the pre-fusion conformation using low-resolution electron microscopy techniques. Thus, a stable pre-fusion Class III fusion protein of the invention may be at least 10, 20, 30, 40, 50, 60, 70, 80, or 90% more stable than the non-mutated pre-fusion Class III fusion protein from which it is derived. Percentage stability might be defined in a number of manners: non-limiting examples would include the additional proportion of the stabilised protein, as compared to the wild-type protein, adopting pre-fusion conformation under given conditions, or alternatively a percentage change in the energetic favourability of the pre-fusion to post-fusion conformational change.

The post-fusion conformation of the Class III fusion proteins all have a trimerisation interface that can be readily identified, for example by examination of published crystal structures. Within this post-fusion trimerisation interface, a central extended helix is conserved across all Class III fusion proteins. As such, one of skill in the art will readily be able to identify the amino acids residues of the primary amino acid structure which form the central extended helix in the post-fusion conformation trimerisation interface. Thus, reference to the central extended helix of the post-fusion conformation trimerisation interface herein may refer to the central extended helix of the post-fusion conformation trimerisation interface of any Class III fusion protein. By way of non-limiting example, the central extended helix of the post-fusion conformation trimerisation interface corresponds to: (i) the extended helix C in RVG as described by Yang et al. Cell Host & Microbe 27, 1-13 (2020) (ii) helix F lying within the trimerisation domain of VSVG (Roche et al. Science, 2007); and/or (iii) helix α-C lying within the trimerisation domain of EBV gB as described in Backovic et al. Proc. Natl Acad Sci USA 106(8), 2880-2885 (2009). In VSVG and RVG, the central extended helix in the post-fusion conformation trimerisation interface forms the inner three helices of an anti-parallel six-helix bundle. Thus, references to the Class III fusion protein domain structure, and particularly the post-fusion conformation trimerisation interface, the central extended helix of the post-fusion conformation trimerisation interface and/or the pre-fusion helix which extends into the post-fusion helix may be defined by reference to the nomenclature used in the art (by reference to the above articles by Yang, Backovic and Roche) for the Class III fusion protein in question. Preferably, the protein or immunogenic fragment thereof of the invention comprises one or more mutations that prevent formation of the central extended helix in the post-fusion conformation trimerisation interface, wherein said central extended helix in the post-fusion conformation trimerisation interface corresponds to helix C in RVG.

The protein or immunogenic fragment thereof of the invention may be derived from any Class III fusion protein. By way of non-limiting example, the Class III fusion protein may be derived from a rhabdovirus, a herpesvirus or a baculovirus. Typically, the Class III fusion protein is the glycoprotein protein of a rhabdovirus, the glycoprotein B (gB) of a herpesvirus, or the glycoprotein 64 (gp64) of a baculovirus. Preferably, the Class III fusion protein is the rabies virus glycoprotein (RVG), vesicular stomatitis virus glycoprotein (VSVG), a herpes virus gB (e.g. a cytomegalovirus, Epstein-Barr virus, HSV-1, HSV-2, varicella zoster virus (VZV), HHV-6A, HHV-6B, HHV-7 and/or Kaposi’s sarcoma-associated herpesvirus (HHV-8) gB). More preferably, the Class III fusion protein is a RVG, HSV 1 or HSV 2 gB, CMV gB, EBV gB or VSV G, even more preferably RVG. The skilled person will understand that the Class III fusion protein from which a protein or immunogenic fragment of the invention is derived, may be any naturally or non-naturally occurring Class III fusion. For example, the Class III fusion protein may be a non-naturally occurring chimera of two naturally occurring Class III fusion protein sequences. By way of example, the RVG according to SEQ ID NO: 2 is a chimera produced from two strains of RVG (a WT ectodomain of Pasteur strain G and a WT intra-virion domain of SAD B19 strain G).

Class III fusion proteins comprise an N-terminal signal peptide which can readily be determined from the primary amino acid sequence by the skilled person. Thus, reference to a Class III fusion protein encompasses both Class III fusion protein sequences including the signal peptide and Class III fusion protein sequences excluding the signal peptide sequence. By way of non-limiting example, protein or immunogenic fragment of the invention may be derived from the full length RVG sequence (i.e. including the signal peptide) according to SEQ ID NO: 2, or the RVG sequence excluding the signal peptide according to SEQ ID NO: 3. Typically where specific amino acid residues or regions with a Class III fusion protein are identified by their position herein, these positions are given with respect to the Class III fusion protein sequences excluding any signal peptide, unless otherwise stated. One of skill in the art will readily appreciate that the corresponding position in the sequence including the signal peptide may be simply determined by adding on the length of the signal peptide to the position of the amino acid of interest. By way of non-limiting example, RVG has an 18 amino acid N-terminal signal peptide. Therefore, position 270 of RVG defined relative to the RVG sequence excluding the signal peptide corresponds to position 288 (270 + 18) of the RVG sequence including the signal peptide.

A protein, or immunogenic fragment thereof, of the invention may comprise one or more, two or more, three or more, four or more, five or more, six or more, seven or more, eight or more, nine or more, ten or more, or more mutations relative to the Class III fusion protein from which it is generated, provided that said mutation(s), either independently or in combination, prevent formation of the central extended helix in the post-fusion conformation trimerisation interface.

The primary amino acid structures of all Class III fusion protein share a conserved cysteine residue within the central extended helix of the post-fusion conformation trimerisation interface. This cysteine residue forms a disulphide bond with another cysteine in the N-terminal part of the protein. Whilst these two cysteine residues are distant to each other in the primary structure, the tertiary structure of the post-fusion conformation of the Class III fusion proteins brings these two residues into close proximity. By aligning Class III fusion protein amino acid sequences using this conserved cysteine residue within the central extended helix, the central extended helix of the post-fusion conformation trimerisation interface of any Class III fusion protein can be identified.

Accordingly, the central extended helix of the post-fusion conformation trimerisation interface may be defined as extending, at its N-terminal end, up to the 32^{nd} amino acid residue N-terminal to the conserved cysteine residue of the Class III fusion protein and, at its C-terminal end, up to the 16^{th} amino acid residue C-terminal to the conserved cysteine residue of the Class III fusion protein. These positions may be defined by reference to published crystallographic structures of the post-fusion conformations as described herein. The inventors have surprisingly discovered that one or more mutations in this region can stabilise the pre-fusion conformation of a Class III fusion protein.

The conserved cysteine residue within Class III fusion proteins may correspond to or align with the cysteine residue at amino acid position 283 of the RVG sequence according to SEQ ID NO: 3, the cysteine residue at amino acid position 284 of the VSVG sequence according to SEQ ID NO: 5, the cysteine residue at amino acid position 484 of the EBV gB sequence according to SEQ ID NO: 6 (which includes a signal peptide), the cysteine residue at amino acid position 507 of the CMV gB sequence according to SEQ ID NO: 8 (which includes a signal peptide), the cysteine residue at amino acid position 529 of the HSV-1 gB sequence according to SEQ ID NO: 10 and/or the cysteine residue at amino acid position 526 of the HSV-2 gB sequence according to SEQ ID NO: 32.

Typically, the central extended helix of the post-fusion conformation trimerisation interface may be defined as corresponding to, or aligning with: (i) amino acid residues 262 to 293 of the RVG sequence of SEQ ID NO: 3; (ii) amino acid residues 263 to 294 of the VSVG sequence of SEQ ID NO: 5; (iii) amino acid residues 458 to 500 of the EBV gB sequence of SEQ ID NO: 6; (iv) amino acid residues 481 to 520 of the CMV gB sequence of SEQ ID NO: 8; and/or (v) amino acid residues 503 to 545 of the HSV gB sequence of SEQ ID NO: 10.

A stable pre-fusion Class III fusion protein, or immunogenic fragment thereof of the invention may not contain mutations in any other domains of the fusion protein, including, but not limited to domain I, domain V and/or the fusion loops. Alternatively, a stable pre-fusion Class III fusion protein, or immunogenic fragment thereof of the invention may contain mutations in other domains of the fusion protein, such as domain I, domain V and/or the fusion loops, but these mutations are secondary to the present invention, which requires one or more mutations that prevent formation of the central extended helix in the post-fusion conformation trimerisation interface, wherein said one or more mutation is typically found within the central extended helix in the post-fusion conformation trimerisation interface, as described herein).

The expression “one or more mutation” is intended to encompass one or more amino acid mutation in the primary amino acid sequence of the central extended helix of the post-fusion conformation trimerisation interface when compared to the WT primary amino acid sequence of the Class III fusion protein from which it is derived. In accordance with the present invention, the one or more amino acid mutation results in the stabilisation of the pre-fusion conformation of the Class III fusion protein, or immunogenic fragment thereof compared with the corresponding WT protein. The stable pre-fusion Class III fusion proteins, or immunogenic fragments thereof of the invention typically comprise, or display, one or more epitope that is specific to the pre-fusion conformation of the Class III fusion protein. An epitope that is specific to the pre-fusion conformation is an epitope that is not present in the post-fusion conformation of the Class III fusion protein. Without wishing to be bound by any particular theory, it is believed that the pre-fusion confirmation of Class III fusion proteins contains epitopes that are the same as those on the protein found on natural circulating virions. Thus, the stabilised pre-fusion Class III fusion proteins of the invention are suitable for eliciting the generation of neutralising antibodies against the pre-fusion conformation of Class III fusion proteins.

The one or more mutation that disrupts formation of the central extended helix of the post-fusion conformation trimerisation interface may be more precisely defined as being within a region which corresponds to or aligns with: (i) amino acids residues 262 to 280 of the RVG sequence of SEQ ID NO: 3; (ii) amino acids residues 263 to 281 of the VSVG sequence of SEQ ID NO: 5; (iii) amino acids residues 463 to 481 of the EBV gB sequence of SEQ ID NO: 6; (iv) amino acids residues 486 to 504 of the CMV gB sequence of SEQ ID NO: 8; and/or (v) amino acids residues 508 to 526 of the HSV-1 gB sequence of SEQ ID NO: 10.

The one or more mutation that prevents formation of the central extended helix of the post-fusion conformation trimerisation interface may be more precisely defined as being within a region which corresponds to or aligns with: (i) amino acids residues 267 to 275 of the RVG sequence of SEQ ID NO: 3; (ii) amino acids residues 268 to 276 of the VSVG sequence of SEQ ID NO: 5; (iii) amino acids residues 468 to 476 of the EBV gB sequence of SEQ ID NO: 6; (iv) amino acids residues 491 to 499 of the CMV gB sequence of SEQ ID NO: 8; and/or (v) amino acids residues 513 to 521 of the HSV-1 gB sequence of SEQ ID NO: 10.

The one or more mutation that prevents formation of the central extended helix of the post-fusion conformation trimerisation interface may be even more precisely defined as being within a region which corresponds to or aligns with: (i) amino acids residues 268 to 274 of the RVG sequence of SEQ ID NO: 3; (ii) amino acids residues 269 to 275 of the VSVG sequence of SEQ ID NO: 5; (iii) amino acids residues 469 to 475 of the EBV gB sequence of SEQ ID NO: 6; (iv) amino acids residues 492 to 498 of the CMV gB sequence of SEQ ID NO: 8; and/or (v) amino acids residues 514 to 520 of the HSV-1 gB sequence of SEQ ID NO: 10.

Preferably, the one or more mutation that prevents formation of the central extended helix of the post-fusion conformation trimerisation interface may be more precisely defined as being within a region which corresponds to or aligns with: (i) amino acids residues 270 to 272 of the RVG sequence of SEQ ID NO: 3; (ii) amino acids residues 271 to 273 of the VSVG sequence of SEQ ID NO: 5; (iii) amino acids residues 471 to 473 of the EBV gB sequence of SEQ ID NO: 6; (iv) amino acids residues 494 to 496 of the CMV gB sequence of SEQ ID NO: 8; and/or (v) amino acids residues 516 to 518 of the HSV-1 gB sequence of SEQ ID NO: 10.

The one of more mutation in the central extended helix of the post-fusion conformation trimerisation interface may be any amino acid mutation within the primary amino acid structure of the central extended helix of the post-fusion conformation trimerisation interface that stabilises the pre-fusion conformation of the Class III fusion protein, such as an amino acid substitution, deletion or insertion. In embodiments wherein the protein, or immunogenic fragment thereof, of the invention comprises more than one mutation, each mutation may be independently selected from an amino acid substitution, deletion or insertion.

Typically, the one or more mutation in the central extended helix of the post-fusion conformation trimerisation interface is an amino acid substitution. In other words, the amino acid at a specified position within the central extended helix of the post-fusion conformation trimerisation interface is substituted by a naturally occurring or non-naturally occurring amino acid that is different to the amino acid present at that position in the central extended helix of the post-fusion conformation trimerisation interface from the WT Class III fusion protein from which the protein, or immunogenic fragment thereof, of the invention is derived.

Amino acids are, in principle, divided into different physicochemical groups. Aspartate and glutamate belong to the negatively-charged amino acids. Histidine, arginine and lysine belong to the positively-charged amino acids. Asparagine, glutamine, serine, threonine, cysteine and tyrosine belong to the polar amino acids. Glycine, alanine, valine, leucine, isoleucine, methionine, proline, phenylalanine and tryptophan belong to the non-polar amino acids. Aromatic side groups are to be found among the amino acids, histidine, phenylalanine, tyrosine and tryptophan. A conservative amino acid substitution refers to the replacement of an amino acid with an amino acid having similar physicochemical properties, i.e. belonging to the same physicochemical group as the amino acid to be replaced. In contrast, a non-conservative amino acid substitution refers to the replacement of an amino acid with an amino acid having different physicochemical properties, i.e. belonging to a different physicochemical group as the amino acid to be replaced. Thus, as a non-limiting example, a conservative substitution may involve the substitution of a non-polar amino acid by another non-polar amino acid, such as substituting leucine with isoleucine. As another non-limiting example, a non-conservative substitution may involve the substation of a non-polar amino acid (e.g. leucine) with a negatively-charged amino acid (e.g. aspartate), a positively-charged amino acid (e.g. arginine), or a polar amino acid (e.g. asparagine). In embodiments wherein the protein or immunogenic fragment thereof of the invention comprises more than one amino acid substitution, each amino acid substitution may be independently selected from a non-conservative or conservative amino acid substitution. In a preferred embodiment of the invention, the amino acid substitution is a non-conservative amino acid substitution.

The non-conservative amino acid substitution within the central extended helix of the post-fusion conformation trimerisation interface may be any non-conservative amino acid substitution which provides the necessary physicochemical properties to stabilise the pre-fusion conformation of the Class III fusion protein. By way of non-limiting example, the non-conservative amino acid substitution may be the substitution of histidine, leucine, valine, isoleucine, asparagine, glutamine, tyrosine and/or arginine with proline. Alternatively, the non-conservative amino acid substitution may be the substitution of histidine, proline and/or glutamine with leucine, alanine, isoleucine and/or valine. In preferred embodiments the non-conservative amino acid substitution is selected from: (i) the substitution of histidine, leucine and/or valine with proline; and/or (ii) the substitution of histidine with leucine.

Mutations in the central extended helix of the post-fusion conformation trimerisation interface stabilise the pre-fusion conformation of Class III fusion proteins by preventing conformational change of the Class III fusion protein, for example, through: increasing the rigidity of the pre-fusion conformation; and/or manipulation of the hydrophobicity of pre-fusion conformation. The one or more mutation may be a mutation which disrupts or prevents α-helical secondary structure and so disrupts or prevents the formation of the central extended helix in trimerisation interface of the post-fusion conformation. Preferred examples of residues which may be mutated in this manner include residues 270, 271 and/or 272 of the RVG sequence of SEQ ID NO: 3, or in other Class III fusion proteins, amino acid residues which correspond to or align with one or more these residues within the central extended helix in the post-fusion conformation trimerisation interface, as described herein.

When the protein or immunogenic fragment thereof of the invention comprises more than one mutation in the central extended helix of the post-fusion conformation trimerisation interface, each mutation may be independently selected from any of the mutations or specific examples thereof, for example a mutation which disrupts α-helical secondary structure and so disrupts or prevents the formation of the central extended helix in trimerisation interface of the post-fusion conformation or a an amino acid substitution by an amino acid with increased hydrophobicity compared with the amino acid being substituted. By way of non-limiting example, the protein or immunogenic fragment thereof of the invention may comprise: (i) one or more mutation(s) which disrupts α-helical secondary structure and so disrupts or prevents the formation of the central extended helix in trimerisation interface of the post-fusion conformation; (ii) one or more amino acid substitutions by an amino acid with increased hydrophobicity compared with the amino acid being substituted or (iii) at least one mutation which disrupts α-helical secondary structure and so disrupts or prevents the formation of the central extended helix in trimerisation interface of the post-fusion conformation and at least one amino acid substitution by an amino acid with increased hydrophobicity compared with the amino acid being substituted.

Preferably, the mutation which disrupts α-helical secondary structure and so disrupts or prevents the formation of the central extended helix in trimerisation interface of the post-fusion conformation is an amino acid substitution. Even more preferably, the mutation which disrupts α-helical secondary structure and so disrupts or prevents the formation of the central extended helix in trimerisation interface of the post-fusion conformation is a non-conservative amino acid substitution. Most preferably, the mutation which disrupts α-helical secondary structure and so disrupts or prevents the formation of the central extended helix in trimerisation interface of the post-fusion conformation is a non-conservative amino acid substitution by proline. By way of non-limiting example, a mutation which disrupts α-helical secondary structure and so disrupts or prevents the formation of the central extended helix in trimerisation interface of the post-fusion conformation may be a substitution of histidine, leucine, valine, isoleucine, asparagine, glutamine, tyrosine and/or arginine with proline. Most preferably, the mutation which disrupts α-helical secondary structure and so disrupts or prevents the formation of the central extended helix in trimerisation interface of the post-fusion conformation is a substitution of histidine, leucine and/or valine with proline.

An amino acid substitution by an amino acid with increased hydrophobicity compared with the amino acid being substituted may be any non-conservative amino acid substitutions that results in an increase in the hydrophobicity of the specific amino acid residue. Thus, in a preferred embodiment of the invention, the amino acid substitution by an amino acid with increased hydrophobicity compared with the amino acid being substituted is a substitution of histidine, proline and/or glutamine with leucine, alanine, isoleucine and/or valine. Most preferably, the substitution of is a substitution of histidine with leucine.

The invention provides a stable pre-fusion Class III fusion protein, or an immunogenic fragment thereof, comprising one or more non-conservative amino acid substitutions (preferably to proline) within its pre-fusion conformation central helix. Said one or more non-conservative amino acid substitutions (preferably to proline) prevent formation of the central extended helix in the post-fusion conformation trimerisation interface (as defined herein). By way of non-limiting example, the invention provides a stable pre-fusion Class III fusion protein, or an immunogenic fragment thereof, comprising one or more non-conservative amino acid substitutions (preferably to proline) within its pre-fusion conformation central helix, that prevent formation of the central extended helix in the post-fusion conformation trimerisation interface corresponding to or aligning with: (a) the extended helix C in rabies virus glycoprotein (RVG); (b) helix F of the trimerisation domain of vesicular stomatitis virus glycoprotein (VSVG); and/or (c) helix alpha-C of the trimerisation domain of Epstein-Barr virus glycoprotein B (EBV gB). By way of further non-limiting example, the invention provides a stable pre-fusion Class III fusion protein, or an immunogenic fragment thereof, comprising one or more non-conservative amino acid substitutions (preferably to proline) within its pre-fusion conformation central helix, that prevent formation of the central extended helix in the post-fusion conformation trimerisation interface corresponding to or aligning with amino acid residues: (i) 262 to 293 of the RVG sequence of SEQ ID NO: 3; (ii) 263 to 294 of the VSVG sequence of SEQ ID NO: 5; (iii) 458 to 500 of the EBV gB sequence of SEQ ID NO: 6; (iv) 481 to 520 of the CMV gB sequence of SEQ ID NO: 8; and/or (v) 503 to 545 of the HSV-1 gB sequence of SEQ ID NO: 10. In other words, the invention provides a stable pre-fusion Class III fusion protein, or an immunogenic fragment thereof, comprising one or more non-conservative amino acid substitutions (preferably to proline) within amino acid residues corresponding to or aligning with amino acid residues: (i) 262 to 293 of the RVG sequence of SEQ ID NO: 3; (ii) 263 to 294 of the VSVG sequence of SEQ ID NO: 5; (iii) 458 to 500 of the EBV gB sequence of SEQ ID NO: 6; (iv) 481 to 520 of the CMV gB sequence of SEQ ID NO: 8; and/or (v) 503 to 545 of the HSV-1 gB sequence of SEQ ID NO: 10.

The invention further provides a stable pre-fusion Class III fusion protein, or an immunogenic fragment thereof, comprising one or more non-conservative amino acid substitutions (preferably to proline) within amino acid residues corresponding to or aligning with amino acid residues: (i) amino acids residues 262 to 280 of the RVG sequence of SEQ ID NO: 3; (ii) amino acids residues 263 to 281 of the VSVG sequence of SEQ ID NO: 5; (iii) amino acids residues 463 to 481 of the EBV gB sequence of SEQ ID NO: 6; (iv) amino acids residues 486 to 504 of the CMV gB sequence of SEQ ID NO: 8; and/or (v) amino acids residues 508 to 526 of the HSV-1 gB sequence of SEQ ID NO: 10.

The invention further provides a stable pre-fusion Class III fusion protein, or an immunogenic fragment thereof, comprising one or more non-conservative amino acid substitutions (preferably to proline) within amino acid residues corresponding to or aligning with amino acid residues: (i) amino acids residues 267 to 275 of the RVG sequence of SEQ ID NO: 3; (ii) amino acids residues 268 to 276 of the VSVG sequence of SEQ ID NO: 5; (iii) amino acids residues 468 to 476 of the EBV gB sequence of SEQ ID NO: 6; (iv) amino acids residues 491 to 499 of the CMV gB sequence of SEQ ID NO: 8; and/or (v) amino acids residues 513 to 521 of the HSV-1 gB sequence of SEQ ID NO: 10.

The invention further provides a stable pre-fusion Class III fusion protein, or an immunogenic fragment thereof, comprising one or more non-conservative amino acid substitutions (preferably to proline) within amino acid residues corresponding to or aligning with amino acid residues: (i) amino acids residues 268 to 274 of the RVG sequence of SEQ ID NO: 3; (ii) amino acids residues 269 to 275 of the VSVG sequence of SEQ ID NO: 5; (iii) amino acids residues 469 to 475 of the EBV gB sequence of SEQ ID NO: 6; (iv) amino acids residues 492 to 498 of the CMV gB sequence of SEQ ID NO: 8; and/or (v) amino acids residues 514 to 520 of the HSV-1 gB sequence of SEQ ID NO: 10.

Preferably, the invention further provides a stable pre-fusion Class III fusion protein, or an immunogenic fragment thereof, comprising one or more non-conservative amino acid substitutions (preferably to proline) within amino acid residues corresponding to or aligning with amino acid residues: (i) amino acids residues 270 to 272 of the RVG sequence of SEQ ID NO: 3; (ii) amino acids residues 271 to 273 of the VSVG sequence of SEQ ID NO: 5; (iii) amino acids residues 471 to 473 of the EBV gB sequence of SEQ ID NO: 6; (iv) amino acids residues 494 to 496 of the CMV gB sequence of SEQ ID NO: 8; and/or (v) amino acids residues 516 to 518 of the HSV-1 gB sequence of SEQ ID NO: 10.

The protein or immunogenic fragment thereof of the invention may have an amino acid substitution at one or more amino acid residue corresponding to or aligning with amino acid residue 270, 271, 272, and/or 273 of the RVG sequence according to SEQ ID NO: 3. Preferably, the one or more amino acid residue corresponding to or aligning with amino acid residue 270, 271, 272 and/or 273 of the RVG sequence according to SEQ ID NO: 3 is substituted by proline. Particularly preferred is the substitution of the amino acid residue corresponding to or aligning with amino acid residue 270 of the RVG sequence according to SEQ ID NO: 3 by proline.

The protein or immunogenic fragment thereof of the invention may have an amino acid substitution at one or more amino acid residue corresponding to or aligning with amino acid residue 271, 272, 273, and/or 274 of the VSVG sequence according to SEQ ID NO: 5. Preferably, the one or more amino acid residue corresponding to or aligning with amino acid residue 271, 272, 273 and/or 274 of the VSVG sequence according to SEQ ID NO: 5 is substituted by proline. Particularly preferred is the substitution of the amino acid residue corresponding to or aligning with amino acid residue 271 of the VSVG sequence according to SEQ ID NO: 5 by proline.

The protein or immunogenic fragment thereof of the invention may have an amino acid substitution at one or more amino acid residue corresponding to or aligning with amino acid residue 471, 472, 473, and/or 474 of the EBV gB sequence according to SEQ ID NO: 6. Preferably, the one or more amino acid residue corresponding to amino acid residue 471, 472, 473 and/or 474 of the EBV gB sequence according to SEQ ID NO: 6 is substituted by proline. Particularly preferred is the substitution of the amino acid residue corresponding to or aligning with amino acid residue 471 of the EBV gB sequence according to SEQ ID NO: 6 by proline.

The protein or immunogenic fragment thereof of the invention may have an amino acid substitution at one or more amino acid residue corresponding to or aligning with amino acid residue 494, 495, 496 and/or 497 of the CMV gB sequence according to SEQ ID NO: 8. Preferably, the one or more amino acid residue corresponding to or aligning with amino acid residue 494, 495, 496 and/or 497 of the CMV gB sequence according to SEQ ID NO: 8 is substituted by proline. Particularly preferred is the substitution of the amino acid residue corresponding to or aligning with amino acid residue 494 of the CMV gB sequence according to SEQ ID NO: 8 by proline.

The protein or immunogenic fragment thereof of the invention may have an amino acid substitution at one or more amino acid residue corresponding to or aligning with amino acid residue 516, 517, 518, and/or 519 of the HSV-1 gB sequence according to SEQ ID NO: 10. Preferably, the one or more amino acid residue corresponding to or aligning with amino acid residue 516, 517, 518 and/or 519 of the HSV-1 gB sequence according to SEQ ID NO: 10 is substituted by proline. Particularly preferred is the substitution of the amino acid residue corresponding to or aligning with amino acid residue 517 of the HSV-1 gB sequence according to SEQ ID NO: 10 by proline.

In some preferred embodiments of the invention, the protein or immunogenic fragment thereof is the RVG sequence (for example an RVG sequence corresponding to SEQ ID NO: 3) comprising one or more amino acid substitutions selected from H270P, L271P, V272P, and/or V273P. Typically, the protein or immunogenic fragment thereof of the invention is the RVG sequence (e.g. an RVG sequence corresponding to SEQ ID NO: 3) comprising: (i) a H270P substitution; (ii) a L271P substitution; (iii) a V272P substitution; (iv) a V273P substitution; (vi) a H270P and L271P substitution; (vii) a H270P and V272P substitution; (viii) a H270P and V273P substitution; (x) a L271P and V272P substitution; (xi) an L271P and V273P substitution; (xiii) a V272P and V273P substitution; (xvi) a H270P, L271P and V272P substitution; (xviii) a H270P, V272P and V273P substitution; (xxi) a L271P, V272P and V273P substitution; or (xxv) a H270P, L271P, V272P and V273P substitution;. Preferably, the protein or immunogenic fragment thereof is the RVG sequence corresponding to SEQ ID NO: 3 comprising at least an H270P substitution.

In some preferred embodiments of the invention, the protein or immunogenic fragment thereof is the VSVG sequence (for example an VSVG sequence corresponding to SEQ ID NO: 5) comprising one or more amino acid substitutions selected from L271P, I272P, Q273P, and/or D274P. Typically, the protein or immunogenic fragment thereof of the invention is the VSVG sequence (e.g. an VSVG sequence corresponding to SEQ ID NO: 5) comprising: (i) a L271P substitution; (ii) a I272P substitution; (iii) a Q273P substitution; (iv) a D274P substitution; (vi) a L271P and I272P substitution; (vii) a L271P and Q273P substitution; (viii) a L271P and D274P substitution; (x) a I272P and Q273P substitution; (xi) a I272P and D274P substitution; (xiii) a Q273P and D274P substitution; (xvi) a L271P, I272P and Q273P substitution; (xviii) a L271P, Q273P and D274P substitution; (xxi) a I272P, Q273P and D274P substitution; (xxv) a L271P, I272P, Q273P and D274P substitution; . Preferably, the protein or immunogenic fragment thereof is the VSVG sequence corresponding to SEQ ID NO: 5 comprising at least an L271P substitution.

In some preferred embodiments of the invention, the protein or immunogenic fragment thereof is the EBV gB sequence (for example an EBV gB sequence corresponding to SEQ ID NO: 6) comprising one or more amino acid substitutions selected from Q471P, I472P, N473P, and/or R474P. Typically, the protein or immunogenic fragment thereof of the invention is the EBV gB sequence (e.g. an EBV gB sequence corresponding to SEQ ID NO: 6) comprising: (i) an Q471P substitution; (ii) an I472P substitution; (iii) an N473P substitution; (iv) an R474P substitution; (vi) an Q471P and I472P substitution; (vii) an Q471P and N473P substitution; (viii) an Q471P and R474P substitution; (x) an I472P and N473P substitutions; (xi) I472P and R474P substitution; (xiii) an N473P and R474P substitution; (xvi) an Q471P, I472P and N473P substitution; (xviii) an Q471P, N473P and R474P substitution; (xxi) an I472P, N473P and R474P substitution; (xxv) an Q471P, I472P, N473P and R474P substitution. Preferably, the protein or immunogenic fragment thereof is the EBV gB sequence corresponding to SEQ ID NO: 6 comprising at least an Q471P substitution.

In some preferred embodiments of the invention, the protein or immunogenic fragment thereof is the CMV gB sequence (for example a CMV gB sequence corresponding to SEQ ID NO: 8) comprising one or more amino acid substitutions selected from Y494P, I495P, N496P, and/or R497P. Typically, the protein or immunogenic fragment thereof of the invention is the CMV gB sequence (e.g a CMV gB sequence corresponding to SEQ ID NO: 8) comprising: (i) an Y494P substitution; (ii) an I495P substitution; (iii) an N496P substitution; (iv) an R497P substitution; (vi) an Y494P and I495P substitution; (vii) an Y494P and N496P substitution; (viii) an Y494P and R497P substitution; (x) an I495P and N496P substitution; (xi) an I495P and R497P substitution; (xiii) an N496P and R497P substitution; (xvi) an Y494P, I495P and N496P substitution; (xviii) an Y494P, N496P and R497P substitution; (xxi) an I495P, N496P and R497P substitution; (xxv) an Y494P, I495P, N496P and R497P substitution. Preferably, the protein or immunogenic fragment thereof is the CMV gB sequence corresponding to SEQ ID NO: 8 comprising at least an Y494P substitution.

In some preferred embodiments of the invention, the protein or immunogenic fragment thereof is the HSV-1 or HSV-2 gB sequence (for example an HSV-1 gB sequence corresponding to SEQ ID NO: 10) comprising one or more amino acid substitutions selected from H516P, V517P, N518P, and/or D519P, or the equivalent (aligning residues) in HSV-2. Typically, the protein or immunogenic fragment thereof of the invention is the HSV gB sequence (e.g. an HSV-1 gB sequence corresponding to SEQ ID NO: 10) comprising: (i) a H516P substitution; (ii) a V517P substitution; (iii) a N518P substitution; (iv) a D519P substitution; (vi) a H516P and V517P substitution; (vii) a H516P and N518P substitution; (viii) a H516P and D519P substitution; (x) a V517P and N518P substitution; (xi) a V517P and D519P substitution; (xiii) a N518P and D519P substitution; (xvi) a H516P, V517P and N518P substitution; (xviii) a H516P, N518P and D519P substitution; (xxi) a V517P, N518P and D519P substitution; or (xxv) a H516P, V517P, N518P and D519P substitution;. Preferably, the protein or immunogenic fragment thereof is the HSV-1 gB sequence corresponding to SEQ ID NO: 10 comprising at least an H516P substitution.

In some embodiments of the invention, the protein or immunogenic fragment thereof comprises or consists of the amino acid sequence of SEQ ID NO: 11 (RVG with H270P). In some embodiments of the invention, the protein or immunogenic fragment thereof comprises or consists of the amino acid sequence of SEQ ID NO: 12 (RVG with L271P). In some embodiments of the invention, the protein or immunogenic fragment thereof comprises or consists of the amino acid sequence of SEQ ID NO: 13 (RVG with V272P). In some embodiments of the invention, the protein or immunogenic fragment thereof comprises or consists of the amino acid sequence of SEQ ID NO: 14 (RVG with V273P).Preferably, the protein or immunogenic fragment thereof comprises or consists of the amino acid sequence of SEQ ID NO: 11 (RVG with H270P).

In some embodiments of the invention, the protein or immunogenic fragment thereof comprises or consists of the amino acid sequence of SEQ ID NO: 15 (VSVG with L271P). In some embodiments of the invention, the protein or immunogenic fragment thereof comprises or consists of the amino acid sequence of SEQ ID NO: 16 (VSVG with I272P). In some embodiments of the invention, the protein or immunogenic fragment thereof comprises or consists of the amino acid sequence of SEQ ID NO: 17 (VSVG with Q273P). In some embodiments of the invention, the protein or immunogenic fragment thereof comprises or consists of the amino acid sequence of SEQ ID NO: 18 (VSVG with D274P). Preferably, the protein or immunogenic fragment thereof comprises or consists of the amino acid sequence of SEQ ID NO: 15 (VSVG with L271P).

In some embodiments of the invention, the protein or immunogenic fragment thereof comprises or consists of the amino acid sequence of SEQ ID NO: 19 (EBV gB with Q471P). In some embodiments of the invention, the protein or immunogenic fragment thereof comprises or consists of the amino acid sequence of SEQ ID NO: 20 (EBV gB with I472P). In some embodiments of the invention, the protein or immunogenic fragment thereof comprises or consists of the amino acid sequence of SEQ ID NO: 21 (EBV gB with N473P). In some embodiments of the invention, the protein or immunogenic fragment thereof comprises or consists of the amino acid sequence of SEQ ID NO: 22 (EBV gB with R474P). Preferably, the protein or immunogenic fragment thereof comprises or consists of the amino acid sequence of SEQ ID NO: 19 (EBV gB with Q471P).

In some embodiments of the invention, the protein or immunogenic fragment thereof comprises or consists of the amino acid sequence of SEQ ID NO: 23 (CMV gB with Y494P). In some embodiments of the invention, the protein or immunogenic fragment thereof comprises or consists of the amino acid sequence of SEQ ID NO: 24 (CMV gB with I495P). In some embodiments of the invention, the protein or immunogenic fragment thereof comprises or consists of the amino acid sequence of SEQ ID NO: 25 (CMV gB with N496P). In some embodiments of the invention, the protein or immunogenic fragment thereof comprises or consists of the amino acid sequence of SEQ ID NO: 26 (CMV gB with R497P). Preferably, the protein or immunogenic fragment thereof comprises or consists of the amino acid sequence of SEQ ID NO: 23 (CMV gB with Y494P).

In some embodiments of the invention, the protein or immunogenic fragment thereof comprises or consists of the amino acid sequence of SEQ ID NO: 27 (HSV-1 gB with H516P). In some embodiments of the invention, the protein or immunogenic fragment thereof comprises or consists of the amino acid sequence of SEQ ID NO: 28 (HSV-1 gB with V517P). In some embodiments of the invention, the protein or immunogenic fragment thereof comprises or consists of the amino acid sequence of SEQ ID NO: 29 (HSV-1 gB with N518P). In some embodiments of the invention, the protein or immunogenic fragment thereof comprises or consists of the amino acid sequence of SEQ ID NO: 30 (HSV-1 gB with D519P). Preferably, the protein or immunogenic fragment thereof comprises or consists of the amino acid sequence of SEQ ID NO: 27 (HSV-1 gB with H516P).

The present invention also encompasses variants of the protein, or immunogenic fragment thereof, sequences defined above, provided said variants retain the one or more mutation which prevents the central extended helix in the post-fusion conformation trimerisation interface. Such variant proteins, or immunogenic fragments thereof, will have at least 60%, at least 70%, at least 80%, at least 85%, at least 90%, at least 95%, at least 99% or more sequence identity with a sequence selected from SEQ ID NO: 11 to 30. Preferably the protein, or immunogenic fragment thereof, has at least 90%, at least 95%, at least 99% or more sequence identity with a sequence selected from SEQ ID NO: 11 to 30.

The mutant Class III fusion proteins of the invention are stabilised in the pre-fusion form. As used herein, this means that the proteins maintain their pre-fusion conformation (described herein, e.g. length:central width aspect ratio, binding to pre-fusion epitope specific antibodies, etc.) in conditions that would cause the corresponding WT Class III fusion protein to transition into the post-fusion conformation. By way of non-limiting example, a stabilised pre-fusion conformation of RVG according to the invention may maintain at least 50%, at least 60%, at least 70%, at least 80% or more reactivity with a pre-fusion specific antibody (such as 1112-1, RVC20, 17C7 or E559, all of which are well-characterised in the art) at a pH of 6 or less, preferably pH 5.8 or less. Either structural techniques (such as low resolution electron microscopy or crystallography), or non-structural techniques (such as determining reactivity with pre-fusion specific antibodies) as described herein, or a combination of both structural and non-structural techniques, may be used to determine and/or quantify the stability of the pre-fusion conformation of the Class III fusion proteins of the invention. By way of non-limiting example, Si et al. PLOS pathogens 14(12):e1007452 (2018) describe the use of low-resolution cryo electron tomography (cryoET) with Volta phase plate for visualising Class III fusion proteins, particularly for determining the pre- and post-fusion conformation structures.

The mutations described herein may be used individually, in combination, or in addition to other mutations and/or stabilisation strategies in order to provide stabilised pre-fusion conformation proteins or immunogenic fragments of the invention. It will be appreciated that, whilst the mutations described herein stabilise the pre-fusion conformation of Class III fusion proteins compared to the non-mutated Class III fusion protein from which they are derived, the degree of stabilisation may be optimised through: (i) the use of a combination of mutations that prevent formation of the central extended helix in the post-fusion conformation trimerisation interface; and/or (ii) the combination of a mutation(s) described herein together with additional mutations/stabilisation strategies which target regions of the Class III fusion protein other than the central extended helix of the post-fusion conformation trimerisation interface. By way of non-limiting example, stable pre-fusion conformation Class III fusion protein of the invention comprising an H270P mutation may be further stabilised by the addition of a resistant to acid-induced neutralization (RAIN) mutation, for example, a M44V mutation. Optimisation of the Class III fusion protein of this invention may provide improved viral antigens for the production of vaccines.

The protein or immunogenic fragment thereof of the invention may further comprise one or more additional mutations in a region other than the central extended helix of the post-fusion conformation trimerisation interface. Examples of such mutations are known in the art. By way of non-limiting example, additional mutations that may be included in a stabilised pre-fusion conformation of CMV gB according to the invention are described in Chandramouli et al. (Nat. Comm DOI: 10.1038/ncomms9176). The protein or immunogenic fragment thereof of the invention may further comprise at least 1, 2, 3, 4, 5, 6, 7, 8, 9, 10, 11, 12, 13, 14, 15, 16, 17, 18, 19 or 20 additional mutations in a region other than the central extended helix of the post-fusion conformation trimerisation interface. The skilled person can easily determine whether a protein or immunogenic fragment thereof of the invention has an additional mutation, for example, by comparing the primary amino acid sequence of the protein or immunogenic fragment thereof with the primary amino acid sequence of the corresponding WT protein. In embodiments wherein the protein or immunogenic fragment thereof of the invention further comprises more than one additional mutation in a region other than the central extended helix of the post-fusion conformation trimerisation interface, each additional mutation may independently be in any region other than central extended helix of the post-fusion conformation trimerisation interface.

Typically the protein or immunogenic fragment thereof of the invention further comprises an additional mutation at an amino acid residue corresponding to, or aligning with, amino acid residue 261 of the RVG sequence according to SEQ ID NO: 3. The additional mutation is typically an amino acid substitution by an amino acid with increased hydrophobicity compared to the amino acid being substituted. By way of non-limiting example, the amino acid residue corresponding to or aligning with amino acid residue 261 of the RVG sequence according to SEQ ID NO:3 may be substituted by leucine, alanine, isoleucine or valine. Preferably, the amino acid residue corresponding to or aligning with amino acid residue 261 of the RVG sequence according to SEQ ID NO:3 is substituted by leucine.

Thus, in one embodiment wherein the protein or immunogenic fragment thereof is derived from an RVG sequence (such as the RVG sequence according to SEQ ID NO: 3), the at least one additional mutation is a H261L substitution. In one embodiment wherein the protein or immunogenic fragment thereof is derived from a VSVG sequence (such as the VSVG sequence according to SEQ ID NO: 5), the at least one additional mutation is a P261X substitution, wherein X is any amino acid that is more hydrophobic than proline. In one embodiment wherein the protein or immunogenic fragment thereof is derived from an EBV gB sequence (such as the EBV gB sequence according to SEQ ID NO: 6), the at least one additional mutation is a Q462X substitution, wherein X is any amino acid that is more hydrophobic than glutamine. In one embodiment wherein the protein or immunogenic fragment thereof is derived from an CMV gB sequence (such as the CMV gB sequence according to SEQ ID NO: 8), the at least one additional mutation is a Q485X substitution, wherein X is any amino acid that is more hydrophobic than glutamine. In one embodiment wherein the protein or immunogenic fragment thereof is derived from an HSV-1 or HSV-2 gB sequence (such as the HSV-1 gB sequence according to SEQ ID NO: 10), the at least one additional mutation is a Q507X substitution, wherein X is any amino acid that is more hydrophobic than glutamine.

Typically, a Class III fusion protein or immunogenic fragment thereof according to the invention induces neutralising antibodies against one or more epitope of the pre-fusion conformation of a Class III fusion protein. The term “neutralising antibody” is defined herein to mean an antibody which by itself (i.e. in the absence of any other anti-Class III fusion protein antibody) has the ability to affect the function of the Class III fusion protein to which it binds. In particular, neutralising antibodies reduce the ability of viral particles expressing the stabilised Class III fusion protein from infecting a cell by neutralising or inhibiting the biological activity of the fusion protein. For example, the neutralising antibody may inhibit the transition of the Class III fusion protein from the pre-fusion conformation to the post-fusion conformation, or may prevent the fusion protein from binding to a host cell receptor. By way of non-limiting example, the protein or immunogenic fragment thereof according to the invention may be a stabilised pre-fusion conformation of RVG which induces neutralising antibodies against the site I and/or site II epitopes, preferably both the site I and site II epitopes. The site I, II and III epitopes of RVG are well characterised in the art, together with antibodies that bind to these sites (Kuzmina et al. J. Antivirals and Antiretrovirals 2013, 5(2):037-043). Site I is located at positions corresponding to amino acids 226-231 of RVG (e.g. SEQ ID NO: 1). Site II is a discontinuous conformational epitope located at positions corresponding to amino acids 34-42 and 198-200 of RVG (e.g. SEQ ID NO: 1). Site III is located at positions corresponding to amino acids 330-338 of RVG (e.g. SEQ ID NO: 1).

The site I epitope is linear and present in RVG under neutral conditions (i.e. when the RVG is in the pre-fusion conformation). However, the site I epitope is occluded in the post-fusion conformation and not present at acidic pH. Thus, a stabilised class III fusion protein or immunogenic fragment thereof according to the invention may be a stabilised pre-fusion conformation of RVG which induces neutralising antibodies against the site I epitope. The site I epitope may be recognised by monoclonal antibody RVC20 (which is well-characterised in the art, see, for example, De Benedictis et al. EMBO Molecular Medicine 2016, 8(4):407-421).

The site II epitope is also present in RVG under neutral conditions (i.e. when the RVG is in the pre-fusion conformation). However, the site II epitope is not present in the post-fusion conformation. The site II epitope may be recognised by monoclonal antibody 1112-1 (which is commercially available). Well-characterised monoclonal antibody E559 also binds to the pre-fusion RVG site II epitope.

A stabilised Class III fusion protein of the invention may induce enhanced neutralising antibody production, particularly against the site I and/or site II epitopes and/or may stabilise neutralising antibody epitopes on the Class III fusion protein, particularly the site I and/or site II epitopes.

Whilst the site III epitope is conformational, it is not typically discriminative between the pre-and post-fusion conformations. However, binding of antibodies specific to the site III epitope of RVG may serve as a test that the RVG protein or immunogenic fragment thereof is correctly folded. Thus, a stabilised class III fusion the protein or immunogenic fragment thereof according to the invention may be a stabilised pre-fusion conformation of RVG which induces neutralising antibodies against the site III epitope. The site III epitope may be recognised by monoclonal antibody 17C7 (which is well-characterised in the art, see, for example, Kuzmina et al. 2013 supra). Thus, a stabilised Class III fusion protein of the invention may induce enhanced neutralising antibody production, particularly against the site I and/or site II epitopes (preferably both site I and site II) and may also be recognised by antibodies against the site III epitopes. A stabilised Class III fusion protein of the invention may stabilise neutralising antibody epitopes on the Class III fusion protein, particularly the site I and/or site II epitopes (preferably both site I and site II), and may also be recognised by antibodies against the site III epitopes.

Typically, the conformation of a stabilised class III fusion the protein or immunogenic fragment thereof according to the invention is substantially the same as that of the pre-fusion conformation of the corresponding WT class III fusion protein. The conformation of a stabilised class III fusion the protein or immunogenic fragment thereof according to the invention may be determined using antibodies which bind to specific antigenic sites of the protein. By way of non-limiting example, a stabilised class III fusion the protein or immunogenic fragment thereof according to the invention may be recognised by an antibody which binds to antigenic sites I, II and/or III. As indicated above, antibodies that recognise these antigenic sites are well known. For example, the monoclonal antibodies RVC20, 1112-1 and 17C7 (which are all well-characterised and/or commercially available) recognise antigenic sites I, II and III respectively.

In view of the distinct structural characteristics between the pre- and post-fusion conformations of Class III fusion proteins, a skilled person can readily determine which confirmation the Class III fusion protein is in. Suitable techniques for determining the conformation of a Class III fusion protein are routine in the art. By way of non-limiting example, low-resolution structural analysis of Class III fusion proteins has shown that the ectodomain of the pre-fusion conformation of Class III fusion proteins is significantly shorter in length (typically about 3 nm shorter, or about 20% shorter), significantly broader in central width (typically about 3.5 nm broader, or about 50% broader) than the ectodomain of the post-fusion conformation, and significantly broader in the base width (typically about 3 nm broader, or about 86% broader). The length and central width of the two conformations of a Class III fusion protein may be used to determine length:central width aspect ratio for each conformation, with the pre-fusion form having a decreased length:central width ratio compared with the post-fusion form. Various low-resolution methods of assessing the size of viral fusion proteins are known in art, for example, cryo-electron tomography (cryoET).

Thus, in some embodiments, the protein or immunogenic fragment thereof according to the invention has an ectodomain length:central width aspect ratio of less than the ectodomain length:central width aspect ratio of the post-fusion conformation of the corresponding Class III fusion protein. Typically, the protein or immunogenic fragment thereof according to the invention has an ectodomain length:central width aspect ratio of less than 2:1, for example, 1.9:1 or less, 1.8:1 or less, 1.7:1 or less, 1.6:1 or less, 1.5:1 or less, 1.4:1 or less, 1.3:1 or less, or 1.2:1 or less, 1.1:1 or less, or 1:1 or less. Preferably, the protein or immunogenic fragment thereof according to the invention has an ectodomain length:central width aspect ratio within the range of 1.4:1 to 1:1, preferably 1.3:1 to 1:1. Even more preferably, the protein or immunogenic fragment thereof according to the invention has an ectodomain length:central width aspect ratio of about 1.2:1.

Stabilised pre-fusion Class III fusion proteins or immunogenic fragments according to the invention may also be defined in terms of the number of amino acid residues of the central alpha-helix that forms part of the trimerisation interface in the post-fusion conformation and which is disrupted by the stabilising mutations of the invention. In more detail, this central alpha-helix of Class III fusion proteins has fewer amino acid residues in the pre-fusion confirmation as opposed to the post-fusion confirmation. Thus, the protein or immunogenic fragment thereof according to the invention typically has a central alpha-helix comprising fewer amino acids than the central alpha-helix in the post-fusion conformation of the Class III fusion protein from which the protein or immunogenic fragment thereof is derived. Typically the central alpha-helix of the stabilised pre-fusion Class III protein, or immunogenic fragment thereof, will have at least one, at least two, at least three, at least four, at least five, at least six, at least seven, at least eight, at least nine, at least ten, at least eleven, at least twelve, at least 13, at least 14, at least 15, at least 16, at least 17, at least 18, at least 19, at least 20, or more fewer amino acid residues than the central alpha-helix of the post-fusion conformation of the corresponding WT Class III fusion protein. Preferably, the protein or immunogenic fragment thereof according to the invention comprises a central alpha-helix having at least ten to 20 fewer amino acid residues than the central alpha-helix of the post-fusion conformation of the corresponding WT Class III fusion protein. More preferably, the protein, or immunogenic fragment thereof according to the invention comprises a central alpha-helix having at least 12 to 17 fewer amino acid residues than the central alpha-helix of the post-fusion conformation of the corresponding WT Class III fusion protein.

WT Class III fusion proteins typically exist in the pre-fusion conformation at neutral, or basic pH (i.e. around pH 7.0 or above). A decrease in pH (i.e. exposure to acidic conditions, e.g. less than pH 6, less than pH 5.8, less than pH 5.5) can trigger the transition of some proteins to the post-fusion conformation. Due to their stabilising mutations, the proteins or immunogenic fragment thereof of the invention typically retain the pre-fusion conformation even in acidic conditions. Thus, in some embodiments, the protein or immunogenic fragment thereof according to the invention displays pre-fusion conformation characteristics (e.g. specific epitopes and/or ectodomain length:central width aspect ratio) when exposed to acidic conditions, for example, a pH of 6 or less, a pH of 5.9 or less, a pH of 5.8 or less, a pH of 5.7 or less, a pH of 5.6 or less or a pH of 5.5 or less.

The present invention also encompasses variants of the stablished pre-fusion conformation class III fusion proteins as described herein, provided said variants retain one or more mutation which prevents the central extended helix in the post-fusion conformation trimerisation interface. Such a variant stablished pre-fusion conformation class III fusion protein will have at least 60%, at least 70%, at least 80%, at least 85%, at least 90%, at least 95%, at least 99% or more sequence identity with the stablished pre-fusion conformation class III fusion protein from which it is derived. Preferably the stablished pre-fusion conformation class III fusion protein has at least 90%, at least 95%, at least 99% or more sequence identity with the stablished pre-fusion conformation class III fusion protein from which it is derived. The protein or immunogenic fragment thereof of the invention may comprise a leader sequence and/or molecular tag or label. Molecular tags or labels may assist in the detection or purification of the tagged/labelled protein or immunogenic fragment thereof during recombinant production. The molecular tags or labels may also be useful for identification of the protein or immunogenic fragment thereof in in vitro/in vivo studies. Various molecular tag/labels are well known to the skilled person, however, by way of non-limiting examples, the molecular tag or label may be a fluorescent tag, such as green fluorescent protein, a polyhistidine-tag, a FLAG-tag, a Strep-II tag or a glutathione-S-transferase-tag. Typically, the molecular tag or label is provided at the amino-terminus (N-terminus) or carboxy-terminus (C-terminus) of the protein or immunogenic fragment thereof.

The stabilised pre-fusion conformation class III fusion proteins of the invention possess numerous advantages from a manufacturing perspective. For example, the stabilised pre-fusion conformation class III fusion proteins of the invention can be expressed at higher levels during recombinant production compared with their WT counterparts. Without being bound by theory, it is believed that this is because the stabilised forms are more energetically stable, resulting in a lower degree of misfolding during protein production, which increases the level of expression from cells (beneficial for both vectored delivery and for protein manufacturing). In addition, as result of their enhanced stability, the stabilised pre-fusion conformation class III fusion proteins of the invention typically have an increased shelf-life, e.g. degradation and/or aggregation is reduced.

### Polynucleotides and Vectors of the Invention

The present invention also provides a polynucleotide that encodes the protein or immunogenic fragment of the invention. The term polynucleotide encompasses both DNA and RNA sequences.

A polynucleotide of the invention may be used for recombinant expression of the protein or immunogenic fragment of the invention, or as a DNA/RNA vaccine.

A polynucleotide of the invention may optionally be codon optimised for expression in a particular cell type, for example, eukaryotic cells (e.g. mammalian cells, yeast cells, insect cells or plants cells) or prokaryotic cells (e.g. E.coli). The term “codon optimised” refers to the replacement of at least one codon within a base polynucleotide sequence with a codon that is preferentially used by the host organism in which the polynucleotide is to be expressed. Typically, the most frequently used codons in the host organism are used in the codon-optimised polynucleotide sequence. Methods of codon optimisation are well known in the art.

It will be understood by a skilled person that numerous different polynucleotides can encode the same polypeptide as a result of the degeneracy of the genetic code. It is also understood that skilled persons may, using routine techniques, make nucleotide substitutions that do not affect the polypeptide sequence encoded by the nucleic acid molecules to reflect the codon usage of any particular host organism in which the polypeptides are to be expressed. Therefore, unless otherwise specified, a “polynucleotide that encodes the protein or immunogenic fragment of the invention” includes all polynucleotide sequences that are degenerate versions of each other and that encode the same amino acid sequence.

The present invention also provides a vector: (a) comprising a polynucleotide of the invention; and/or (b) encoding a protein or immunogenic fragment thereof of the invention. The vector(s) may be present in the form of a vaccine composition or formulation.

The vector(s) may be a viral vector. Such a viral vector may be an adenovirus (of a human serotype such as AdHu5, a simian serotype such as ChAd63, ChAdOX1 or ChAdOX2, or another form), a poxvirus vector (such as a modified vaccinia Ankara (MVA)), or an adeno associated virus (AAV). ChAdOX1 and ChAdOX2 are disclosed in WO2012/172277 (herein incorporated by reference in its entirety). ChAdOX2 is a BAC-derived and E4 modified AdC68-based viral vector. Preferably said viral vector is an adenovirus.

Viral vectors are usually non-replicating or replication impaired vectors, which means that the viral vector cannot replicate to any significant extent in normal cells (e.g. normal human cells), as measured by conventional means - e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown - for example, modified vaccinia Ankara (MVA) can be grown in CEF cells. In one embodiment, the vector is selected from a human or simian adenovirus or a poxvirus vector

Typically, the viral vector is incapable of causing a significant infection in an animal subject, typically in a mammalian subject such as a human or other primate.

The vector(s) may be a DNA vector, such as a DNA plasmid. The vector(s) may be an RNA vector, such as a mRNA vector or a self-amplifying RNA vector. The DNA and/or RNA vector(s) of the invention may be capable of expression in eukaryotic and/or prokaryotic cells.

Typically, the DNA and/or RNA vector(s) are capable of expression in a cell of a subject, for example, a cell of a mammalian or avian subject to be immunised.

The present invention may be a phage vector, such as an AAV/phage hybrid vector as described in Hajitou et al., Cell 2006; 125(2) pp. 385-398; herein incorporated by reference.

Typically, in the vector(s) the polynucleotide of the invention is operably linked to a suitable promoter. The polynucleotide may also be linked to a suitable terminator sequence. Suitable promoter and terminator sequences are well known in the art.

The choice of promoter will depend on where the ultimate expression of the polynucleotide will take place. In general, constitutive promoters are preferred, but inducible promoters may likewise be used. The construct produced in this manner includes at least one part of a vector, in particular, regulatory elements. The vector is preferably capable of expressing the nucleic acid in a given host cell. Any appropriate host cell may be used, such as mammalian, bacterial, insect, yeast, and/or plant host cells. In addition, cell-free expression systems may be used. Such expression systems and host cells are standard in the art.

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

Vector(s) of the present invention may be designed in silico, and then synthesised by conventional polynucleotide synthesis techniques.

### Virus-Like Particles

Virus-like particles (VLPs) are particles which resemble viruses but do not contain viral nucleic acid and are therefore non-infectious. They commonly contain one or more virus capsid or envelope proteins which are capable of self-assembly to form the VLP. VLPs have been produced from components of a wide variety of virus families (Noad and Roy (2003), Trends in Microbiology, 11:438-444; Grgacic et al., (2006), Methods, 40:60-65). Some VLPs have been approved as therapeutic vaccines, for example Engerix-B (for hepatitis B), Cervarix and Gardasil (for human papilloma viruses).

Accordingly, the invention provides a VLP comprising a protein or immunogenic fragment thereof of the invention.

The skilled person will understand that VLPs can be synthesized through the individual expression of viral structural proteins, which can then self-assemble into the virus-like structure. Combinations of structural capsid proteins from different viruses can be used to create recombinant VLPs. In addition, antigens or immunogenic fragments thereof can be fused to the surface of VLPs. By way of non-limiting example, antigens or immunogenic fragments thereof of the invention may be coupled to a VLP using the SpyCatcher-SpyTag system (as described by Brune & Howarth, Front Immunol (9) 1432, 2018. As a further example, antigens or immunogenic fragments may be fused to rationally-designed particle forming domains, such as those reported by Bale et al (Science, Vol 353, Issue 6297, p389). As a further example, the VLP might be an enveloped VLP, such as those derived from retroviruses, lentiviruses, or other enveloped viruses.

A VLP of the invention may comprise one or more further protein antigen in addition to the stabilised class III protein.

### Antibodies and Binding Compounds

The present invention also provides binding compounds to a stabilised pre-conformation Class III fusion protein, or immunogenic fragment thereof, of the invention.

The binding compound may be an antibody, such as a monoclonal antibody or polyclonal antibody. The binding compounds may be an antigen-binding fragment of a monoclonal or polyclonal antibody, or a peptide which specifically binds to the protein or immunogenic fragment of the invention. By way of non-limiting example, an antigen-binding fragment of the invention may be a Fab, Fab₂, Fv, scFv, tandem scFv, or dAb.

Accordingly, there is provided an antibody, or antigen-binding fragment thereof, that specifically binds to the protein or immunogenic fragment thereof of the invention. Preferably the antibody is a monoclonal antibody.

As described herein, the stabilised pre-fusion conformation of a Class III fusion protein of the invention can give rise to neutralising antibodies against the virus from which the stabilised pre-fusion conformation of a Class III fusion protein is derived. The stabilised pre-fusion conformation of a Class III fusion protein of the invention preferably raises neutralising antibodies that bind to the pre-stabilised conformation of the Class III fusion protein and reduce or inhibit its biological activity (e.g. by preventing the pre-fusion form of the Class III fusion protein from binding to a host cell receptor and/or the transition from pre- to post-fusion form). The effectiveness of the stabilised pre-fusion conformation of a Class III fusion protein of the invention may be quantified using any appropriate technique and measured in any appropriate units. For example, the effectiveness of the stabilised pre-fusion conformation of a Class III fusion protein of the invention may be given in terms of their half maximal effective concentration (EC₅₀), antibody titre stimulated (in terms of antibody units, AU) and/or EC₅₀ in terms of AU. The latter of these gives an indication of the quality of the antibody response stimulated by the stabilised pre-fusion conformation of a Class III fusion protein of the invention. Any appropriate technique may be used to determine the EC₅₀, AU or EC₅₀/AU. Conventional techniques are known in the art.

The amount of antibody produced may be quantified using any appropriate method, with standard techniques being known in the art. For example, the amount of antibody produced may be measured by ELISA in terms of the serum IgG response induced by the stabilised pre-fusion conformation of a Class III fusion protein of the invention. The amount of antibody produced may be given in terms of arbitrary antibody units (AU).

The immune response (or immunogenicity) to a stabilised pre-fusion conformation of a Class III fusion protein of the invention, particularly the antibody response, may be given as the half-maximal effective concentration in terms of the amount of antibody produced, i.e. EC₅₀/AU. This gives an indication of the quality of the immune response generated to the stabilised pre-fusion conformation of a Class III fusion protein. For example, a low EC₅₀ (i.e. effective response) but a high number of antibody units generated is less effective (and gives a higher EC₅₀/AU) than a low EC₅₀ with a low number of antibody units. This value thus indicates the quality of the antibody response by representing the neutralising antibody activity (measured as the EC₅₀) as a proportion of the total amount of anti-Class III fusion protein IgG antibody produced (measured by ELISA in AU). A more effective vaccine thus induces the EC₅₀ with less antibody (lower AU).

Preferably a stabilised pre-fusion conformation of a Class III fusion protein of the invention elicits an improved immune response, particularly an improved antibody response, compared with the Class III protein in its post-fusion conformation, or the Class III protein as present in complete viral particles (where a proportion of the Class III protein will not be in the pre-fusion conformation, but transitioning between different intermediate energy states). For example, a stabilised pre-fusion conformation of a Class III fusion protein of the invention may elicit antibodies with a greater a lower EC₅₀, and/or a lower EC₅₀/AU than the Class III protein in its post-fusion conformation, or the Class III protein as present in complete viral particles.

Alternatively, the binding compound may be an oligonucleotide aptamer. The aptamer may specifically bind to the protein or immunogenic fragment thereof of the invention.

Oligonucleotide aptamers may be identified or synthesised using well-established methods. The aptamer may further me optimised to render is suitable for therapeutic use, e.g. t may be conjugated to a monoclonal antibody to modify its pharmacokinetics and/or recruit Fc-dependent immune functions.

By specific, it will be understood that the binding compound (e.g. antibody, antigen-biding fragment thereof or oligonucleotide aptamer) binds to a stabilised pre-fusion conformation Class III fusion protein of the invention (or fragment thereof), with no significant cross-reactivity to any other molecule, particularly any other protein, and even more particularly the post-fusion conformation of that Class III fusion protein. As a non-limiting example, an antibody that is specific for a stabilised pre-fusion conformation of RVG will show no significant cross-reactivity with a stabilised pre-fusion conformation of EBV gB or the post-fusion conformation of RVG. Cross-reactivity may be assessed by any suitable method. Cross-reactivity of a binding compound of the invention may be considered significant if the binding compound binds to another molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the stabilised pre-fusion conformation Class III fusion protein of the invention (or fragment thereof) against which it is directed. A binding compound that is specific for a stabilised pre-fusion conformation Class III fusion protein of the invention (or fragment thereof) may bind to another molecule at less than 90%, 85%, 80%, 75%, 70%, 65%, 60%, 55%, 50%, 45%, 40%, 35%, 30%, 25% or 20% the strength that it binds to the stabilised pre-fusion conformation Class III fusion protein of the invention (or fragment thereof). Preferably, the binding compound binds to the other molecule at less than 20%, less than 15%, less than 10% or less than 5%, less than 2% or less than 1% the strength that it binds to the stabilised pre-fusion conformation Class III fusion protein of the invention (or fragment thereof).

### Compositions and Therapeutic Indications

As described herein, the present inventors are the first to provide a stabilised pre-fusion conformation of a Class III fusion protein. Said protein, or immunogenic fragment thereof, has utility as a vaccine antigen in view of the stability of the protein and the display of pre-fusion conformation specific epitopes which elicit the production of neutralising antibodies in vivo. The stability of these proteins also provides increased yields during recombinant expression.

Accordingly, the invention provides a protein or immunogenic fragment of the invention for use in a vaccine, particularly a subunit vaccine.

Stabilisation of the pre-fusion conformation of Class III fusion protein in accordance with the present invention provides enhanced display of the epitopes found on the pre-fusion conformation of Class III fusion proteins. By stabilising the pre-fusion conformation, the invention allows enhanced yields of proteins bearing pre-fusion conformation specific epitopes. This enhanced yield, together with long term stability (i.e. during storage) and antigen quality (i.e. the display of the pre-fusion conformation specific epitopes) is advantageous for vaccine production.

As the proteins or immunogenic fragments thereof according to the invention are non-infectious (in so far as they lack viral genomic material) inactivation is not required prior to use in a vaccine.

The invention also provides a vaccine composition comprising a protein or immunogenic fragment thereof according to the invention, and/or a polynucleotide molecule according to the invention, and/or a viral vector and/or DNA plasmid according to the invention, and/or a virus-like according to the invention. The vaccine composition may optionally comprise a pharmaceutically acceptable excipient, diluent, carrier, propellant, salt and/or additive.

Typically, the vaccine composition of the invention is suitable for vaccinating against a virus from which the protein or immunogenic fragment of the invention is derived. By way of a non-limiting example, a vaccine composition comprising a stabilised pre-fusion RVG is suitable for vaccinating against rabies virus.

In view of the common structural architecture amongst Class III fusion proteins a single protein or immunogenic fragment thereof may be suitable for vaccinating against a range of virus types.

In some embodiments the vaccine composition comprises at least two different proteins or immunogenic fragments according to the invention, and/or at least two different polynucleotide molecules according to the invention. By way of non-limiting example, the vaccine composition may comprise a stabilised pre-fusion RVG and a stabilised pre-fusion CMV gB. Typically, vaccine compositions comprising at least two different proteins or immunogenic fragments according to the invention, and/or at least two different polynucleotide molecules according to the invention may be suitable for vaccinating against two different viral types.

Vaccine compositions according to the invention may comprise any combination of stabilised proteins or immunogenic fragments thereof of the invention, and/or different polynucleotide molecules according to the invention, which are derived from the same Class III fusion protein. By way of non-limiting example, a vaccine composition may comprise an RVG comprising the H270P substitution, an RVG comprising the L271P substitution, and an RVG comprising the V272P substitution.

Thus, the invention also provides a protein or immunogenic fragment thereof as defined herein, and/or a vaccine composition as defined herein, and/or the polynucleotide as defined herein, and/or a vector and/or DNA plasmid as defined herein, and/or a virus-like particle as defined herein and/or an antibody as defined herein, for use in a method of immunising a subject against a viral infection. Accordingly, the invention provides a method of treating or preventing a disease caused by a virus comprising a Class III fusion protein by administering a stabilised pre-fusion conformation of said Class III fusion protein as defined herein and/or a vaccine composition as defined herein, and/or the polynucleotide as defined herein, and/or a vector and/or DNA plasmid as defined herein, and/or a virus-like particle as defined herein and/or an antibody as defined herein.

The invention also provides use of a protein or immunogenic fragment thereof as defined herein, and/or a vaccine composition as defined herein, and/or the polynucleotide as defined herein, and/or a vector and/or DNA plasmid as defined herein, and/or a virus-like particle as defined herein and/or an antibody as defined herein, in the manufacture of a medicament for the immunisation of a subject against a viral infection.

As used herein, the term “treatment” or “treating” embraces therapeutic or preventative/prophylactic measures, and includes post-infection therapy and amelioration of a viral infection by a virus comprising a Class III fusion protein.

As used herein, the term “preventing” includes preventing the initiation of a viral infection by a virus comprising a Class III fusion protein and/or reducing the severity or intensity of a viral infection by a virus comprising a Class III fusion protein. The term “preventing” includes inducing or providing protective immunity against a viral infection by a virus comprising a Class III fusion protein. Immunity to a viral infection by a virus comprising a Class III fusion protein may be quantified using any appropriate technique, examples of which are known in the art.

A stabilised pre-fusion conformation of said Class III fusion protein as defined herein and/or a vaccine composition as defined herein, and/or the polynucleotide as defined herein, and/or a vector and/or DNA plasmid as defined herein, and/or a virus-like particle as defined herein and/or an antibody as defined herein may be administered to a subject (typically a mammalian subject such as a human or other primate) already having a viral infection by a virus comprising a Class III fusion protein, a condition or symptoms associated with a viral infection by a virus comprising a Class III fusion protein, to treat or prevent a viral infection by a virus comprising a Class III fusion protein. For example, the subject may be suspected of having come in contact with such a virus, or has had known contact with such a virus, but is not yet showing symptoms of exposure.

When administered to a subject (e.g. a mammal such as a human or other primate) that already has a viral infection by a virus comprising a Class III fusion protein, or is showing symptoms associated with a viral infection by a virus comprising a Class III fusion protein, the stabilised pre-fusion conformation of said Class III fusion protein as defined herein and/or a vaccine composition as defined herein, and/or the polynucleotide as defined herein, and/or a vector and/or DNA plasmid as defined herein, and/or a virus-like particle as defined herein and/or an antibody as defined herein can cure, delay, reduce the severity of, or ameliorate one or more symptoms, and/or prolong the survival of a subject beyond that expected in the absence of such treatment.

Alternatively, a stabilised pre-fusion conformation of said Class III fusion protein as defined herein and/or a vaccine composition as defined herein, and/or the polynucleotide as defined herein, and/or a vector and/or DNA plasmid as defined herein, and/or a virus-like particle as defined herein and/or an antibody as defined herein may be administered to a subject (e.g. a mammal such as a human or other primate) who ultimately may be infected with a virus comprising a Class III fusion protein, in order to prevent, cure, delay, reduce the severity of, or ameliorate one or more symptoms of a viral infection by a virus comprising a Class III fusion protein, or in order to prolong the survival of a subject beyond that expected in the absence of such treatment, or to help prevent that subject from transmitting a viral infection by a virus comprising a Class III fusion protein.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects.

The stabilised pre-fusion conformation of said Class III fusion protein as defined herein and/or a vaccine composition as defined herein, and/or the polynucleotide as defined herein, and/or a vector and/or DNA plasmid as defined herein, and/or a virus-like particle as defined herein and/or an antibody as defined herein may be used in combination with other vaccines or vaccine subunits used to treat viral infections (both the same viral infection as treated by the stabilised pre-fusion conformation of said Class III fusion protein as defined herein and/or a vaccine composition as defined herein, and/or the polynucleotide as defined herein, and/or a vector and/or DNA plasmid as defined herein, and/or a virus-like particle as defined herein and/or an antibody as defined herein; or different viral infections).

The present invention further provides a vaccine composition comprising a Class III fusion protein as defined herein, optionally together with one or more additional antigen or a fragment thereof, where either or both the Class III fusion protein and/or the one or more additional antigen or fragment thereof may be expressed as a soluble recombinant protein. Recombinant protein-based vaccines are well known in the art. They may be, for example, monomeric soluble proteins or soluble fusion proteins. Such proteins are typically administered or formulated in a vaccine adjuvant. Examples of protein-based vaccines are diphtheria and tetanus toxoids.

The Class III fusion protein of the invention and one or more additional antigen or fragment thereof may be combined to provide a single vaccine product (as described above) capable of inducing antibodies against both antigens, e.g. by mixing two separate recombinant protein vaccines, or by co-delivering the antigens using vaccine platforms such as particle-based protein vaccine delivery, or using a fusion of the two antigens; or by using a mixture of viral vectors expressing the individual antigens, or viral vectors co-expressing both antigens.

As used, herein, a “vaccine” is a formulation that, when administered to an animal subject such as a mammal (e.g. a human or other primate) stimulates a protective immune response against a viral infection by a virus comprising a Class III fusion protein. The immune response may be a humoral and/or cell-mediated immune response. A vaccine of the invention can be used, for example, to protect a subject from the effects of a viral infection by a virus comprising a Class III fusion protein.

The viral infection may be any viral infection caused by a virus possessing a Class III fusion protein. By way of non-limiting example, the viral infection may be a herpes virus which infects animals (e.g. monkey B virus, pseudorabies virus, bovine herpesvirus 1, or a avian alpha-herpesviruses, e.g. Marek’s disease virus), rabies, EBLV½, a bat lyssavirus (e.g. Mokola, Duyvenhage, Lagos), Bas-Congo virus or a rhabdoviruses which infects animals (e.g. VSV). Preferably the viral infection is a rabies virus infection, a VSV infection, and/or a herpes virus infection (e.g. a CMV, EBV, HSV-1, HSV-2, varicella zoster virus (VZV), HHV-6A, HHV-6B, HHV-7, and/or Kaposi’s sarcoma-associated herpesvirus (HHV-8) infection).

In the context of therapeutic uses and methods, a “subject” is intended to encompass any animal subject that would benefit from stimulation or induction of an immune response against a protein or immunogenic fragment thereof of the invention. Typically, the subject is a mammalian or avian subject. By way of non-limiting example, the mammalian subject may be a human, non-human primate, pig, cow, sheep, deer, dog, cat, bat or rodent (e.g. mouse, rat, hamster or rabbit). By way of non-limiting example, the avian subject may be a chicken, duck, goose, turkey, emu or ostrich. Preferably the subject is a human.

### Pharmaceutical Compositions and Formulations

The term “vaccine” is herein used interchangeably with the terms “therapeutic/prophylactic composition”, “formulation” or “medicament”.

The vaccine of the invention (as defined above) can be combined or administered in addition to a pharmaceutically acceptable carrier. Alternatively, or in addition, the vaccine of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations (e.g. vaccines) is generally by conventional routes e.g. intravenous, subcutaneous, intraperitoneal, or mucosal routes. The administration may be by parenteral injection, for example, a subcutaneous, intradermal or intramuscular injection. Formulations comprising neutralizing antibodies may be particularly suited to administration intravenously, intramuscularly, intradermally, or subcutaneously.

Accordingly, immunogenic compositions, therapeutic formulations, medicaments and prophylactic formulations (e.g. vaccines) of the invention are typically prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as a Class III fusion protein as defined herein and/or the polynucleotide as defined herein, and/or a vector and/or DNA plasmid as defined herein, and/or a virus-like particle as defined herein and/or an antibody as defined herein) are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/ or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2 % squalene/ Tween 80 emulsion, the MF59 formulation developed by Novartis, and the AS02, AS01, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

### Methods for Identifying Pre-Fusion Conformation Class III Fusion Protein Specific Antibodies

The invention also provides methods for the identification and generation of an antibody, or antigen-binding fragment thereof, specific to an epitope of a pre-fusion conformation Class III fusion protein.

Due to the instability of the pre-fusion conformation of WT Class III fusion proteins, the identification and generation of antibodies specific to the pre-fusion conformation has been challenging. The paucity of antibodies specific to the pre-fusion conformation of Class III fusion proteins has also hampered the research in this field.

Thus, in one embodiment there is provided the use of a protein or immunogenic fragment thereof of the invention for the generation of an antibody, or antigen-binding fragment thereof, specific to an epitope of a pre-fusion conformation Class III fusion protein.

Methods of generating antibodies specific to a particular antigen are well known in the art. A polyclonal antibody may, for example, be generated by injecting an animal (for example, a rabbit or a goat) with a protein, or immunogenic fragment of the invention and subsequently purifying the polyclonal antibody from the antiserum of said animal. Typically, this is followed by screening to identify antibodies with the desired characteristics (here, neutralisation and binding to the pre-fusion conformation of the Class III fusion protein, but not the post-fusion conformation), and optionally epitope mapping. The various methods of generating monoclonal antibodies are also well known in the art and include, for example, the creation and screening of hybridomas, the culture of memory B cells stimulated by cytokines or immortalised by EBV, in vitro display technologies, or single-cell RT-PCR cloning from sorted cells with the desired characteristics.

### DEFINITIONS

The headings provided herein are not limitations of the various aspects or embodiments of this disclosure.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ± 4%, ± 3%, ± 2%, ± 1%, ± 0.5%, ± 0.1%, of the numerical value of the number with which it is being used. Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The term “immunogenic fragment”, when used in relation to a protein of the invention, means a peptide capable of eliciting a neutralising antibody production against said fragment.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Preferably the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of pain. A subject can be male or female, adult or juvenile.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, a subject can be one who exhibits one or more risk factors for a condition or one or more complications related to said condition or a subject who does not exhibit risk factors.

A “subject in need” of treatment for a particular condition can be a subject having that condition, diagnosed as having that condition, or at risk of developing that condition.

As used herein, the term “healthy individual” refers to an individual or group of individuals who are in a healthy state, e.g. individuals who have not shown any symptoms of the disease, have not been diagnosed with the disease and/or are not likely to develop the disease (e.g. a cardiovascular disease or any specific disease described herein). Preferably said healthy individual(s) is not on medication affecting a cardiovascular disease or disorder and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia.

Unless otherwise indicated, any nucleic acid sequences are written left to right in 5’ to 3’ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

A polypeptide, e.g., a fusion polypeptide or portion thereof (e.g. a domain) of the invention, can be a variant of a sequence described herein. Preferably, the variant is a conservative substitution variant. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wild-type, e.g. 110%, 125%, 150%, 175%, 200%, 500%, 1000% or more.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

A polypeptide as described herein may comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

A polypeptide as described herein may comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). A polypeptide as described herein may comprise alternative amino acids. Non-limiting examples of alternative amino acids include D amino acids, beta-amino acids, homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine ), ornithine, citruline, alpha-methyl-alanine, para-benzoylphenylalanine, paraaminophenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, aminoisobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, I-amino-1- cyclopentanecarboxylic acid, I-amino-1-cyclohexanecarboxylic acid, amino-benzoic acid, amino-naphthoic acid, gamma-aminobutyric acid, difluorophenylalanine, nipecotic acid, alphaamino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

A polypeptide may be modified, e.g. by addition of a moiety to one or more of the amino acids comprising the peptide. A polypeptide as described herein may comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. A polypeptide as described herein may comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; endcapping modifications; cyano groups; phosphorylation; albumin, and cyclization.

Alterations of the original amino acid sequence can be accomplished by any of a number of techniques known to one of skill in the art. Amino acid substitutions can be introduced, for example, at particular locations by synthesizing oligonucleotides containing a codon change in the nucleotide sequence encoding the amino acid to be changed, flanked by restriction sites permitting ligation to fragments of the original sequence. Following ligation, the resulting reconstructed sequence encodes an analogue having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. A polypeptide as described herein may be chemically synthesized and mutations can be incorporated as part of the chemical synthesis process.

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

A “fragment” of a polypeptide comprises at least 50%, at least 60%, at least 70%, at least 80%, at least 90%, at least 95%, at least 97% or more of the original polypeptide.

As used herein, the terms “polynucleotide molecule”,“polynucleotide sequence” “polynucleotides”, “nucleic acid” and “nucleic acid sequence” refer to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double- stranded DNA Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA, siRNA, shRNA and antisense oligonucleotides.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| | | |
|---|---|---|
| Amino Acid | Codons | Degenerate Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/ Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more% of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

As used herein the term “comprising” or “comprises” is used in reference to features of products, compositions and methods of the invention, that are essential, yet open to the inclusion of unspecified elements, whether essential or not. Comprising encompasses the term “consisting of”.

The term “consisting of” refers to features of products, compositions and methods of the invention as recited herein, which are exclusive of any element not recited in that description of the invention.

As used herein the term “consisting essentially of” refers to those elements required for a given invention. The term permits the presence of elements that do not materially affect the basic and novel or functional characteristic(s) of that invention.

Embodiments described herein as “comprising” one or more features may also be considered as disclosure of the corresponding embodiments “consisting of” and/or “consisting essentially of” such features.

As used herein, the term “capable of’ when used with a verb, encompasses or means the action of the corresponding verb. For example, “capable of interacting” also means interacting, “capable of cleaving” also means cleaves, “capable of binding” also means binds and “capable of specifically targeting...” also means specifically targets.

### SEQUENCE HOMOLOGY

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-

Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214

; Align-M, see, e.g., Ivo Van Walle et al., Align-M - A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

Alignment score for determining sequence identity

BLOSUM62 table
A R N D C Q E G H I L K M F P S T W Y V
A 4
R -1 5
N -2 0 6
D -2 -2 1 6
C 0 -3 -3 -3 9
Q -1 1 0 0 -3 5
E -1 0 0 2 -4 2 5
G 0 -2 0 -1 -3 -2 -2 6
H -2 0 1 -1 -3 0 0 -2 8
I -1 -3 -3 -3 -1 -3 -3 -4 -3 4
L -1 -2 -3 -4 -1 -2 -3 -4 -3 2 4
K -1 2 0 -1 -3 1 1 -2 -1 -3 -2 5
M -1 -1 -2 -3 -1 0 -2 -3 -2 1 2 -1 5
F -2 -3 -3 -3 -2 -3 -3 -3 -1 0 0-3 0 6
P -1 -2 -2 -1 -3 -1 -1 -2 -2 -3 -3 -1 -2 -4 7
S 1 -1 1 0-1 0 0 0 -1 -2 -2 0 -1 -2 -1 4
T 0 -1 0 -1 -1 -1 -1 -2 -2 -1 -1 -1 -1 -2 -1 1 5
W -3 -3 -4 -4 -2 -2 -3 -2 -2 -3 -2 -3 -1 1 -4 -3 -2 11
Y -2 -2 -2 -3 -2 -1 -2 -3 2 -1 -1 -2 -1 3 -3 -2 -2 2 7
V 0 -3 -3 -3 -1 -2 -2 -3 -3 3 1 -2 1 -1 -2 -2 0 -3 -1 4

The percent identity is then calculated as:

$$\frac{\text{Total number of identical matches}}{\begin{matrix}\left[\text{length of the longer sequence plus the number of gaps introduced}\right) \\ \left(\text{into the longer sequence in order to align the two sequences}\right]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| serine | |
| threonine | |
| methionine | |

In addition to the 20 standard amino acids, non-standard amino acids (such as 4-hydroxyproline, 6-N-methyl lysine, 2-aminoisobutyric acid, isovaline and a -methyl serine) may be substituted for amino acid residues of the polypeptides of the present invention. A limited number of non-conservative amino acids, amino acids that are not encoded by the genetic code, and unnatural amino acids may be substituted for clostridial polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allothreonine, methyl-threonine, hydroxy-ethylcysteine, hydroxyethylhomo-cysteine, nitroglutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90: 10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241 :53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30: 10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### SEQUENCE INFORMATION

Where an initial Met amino acid residue or a corresponding initial codon is indicated in any of the following SEQ ID NOs, said residue/codon may be optional.

Full-length non-chimeric wild-type RVG (Pasteur strain) (Uniprot P08667), including the N-terminal signal peptide: SEQ ID NO: 1

MVPQALLFVP LLVFPLCFGK FPIYTIPDKL GPWSPIDIHH LSCPNNLWE DEGCTNLSGF 60
SYMELKVGYI SAIKMNGFTC TGWTEAETY TNFVGYVTTT FKRKHFRPTP DACRAAYNWK 120
MAGDPRYEES LHNPYPDYHW LRTVKTTKES LVIISPSVAD LDPYDRSLHS RVFPGGNCSG 180
VAVSSTYCST NHDYTIWMPE NPRLGMSCDI FTNSRGKRAS KGSETCGFVD ERGLYKSLKG 240
ACKLKLCGVL GLRLMDGTWV AMQTSNETKW CPPGQLVNLH DFRSDEIEHL WEELVKKRE 300
ECLDALESIM TTKSVSFRRL SHLRKLVPGF GKAYTIFNKT LMEADAHYKS VRTWNEIIPS 360
KGCLRVGGRC HPHVNGVFFN GIILGPDGNV LIPEMQSSLL QQHMELLVSS VIPLMHPLAD 420
PSTVFKNGDE AEDFVEVHLP DVHERISGVD LGLPNWGKYV LLSAGALTAL MLIIFLMTCW 480
RRVNRSEPTQ HNLRGTGREV SVTPQSGKII SSWESYKSGG ETGL 524

The signal peptide is underlined and may be omitted from any polypeptide of the invention.

RVG produced from two strains of RVG (a WT ectodomain of Pasteur strain G and a WT intra-virion domain of SAD B19 strain G) including the N-terminal signal peptide: SEQ ID NO: 2

MVPQALLFVP LLVFPLCFGK FPIYTIPDKL GPWSPIDIHH LSCPNNLWE DEGCTNLSGF 60
SYMELKVGYI SAIKMNGFTC TGWTEAETY TNFVGYVTTT FKRKHFRPTP DACRAAYNWK 120
MAGDPRYEES LHNPYPDYHW LRTVKTTKES LVIISPSVAD LDPYDRSLHS PVFPGGNCSG 180
VAVSSTYCST NHDYTIWMPE NPRLGMSCDI FTNSRGKRAS KGSETCGFVD ERGLYKSLKG 240
ACKLKLCGVL GLRLMDGTWV AMQTSNETKW CPPGQLVNLH DFRSDEIEHL WEELVKKRE 300
ECLDALESIM TTKSVSFRRL SHLRKLVPGF GKAYTIFNKT LMEADAHYKS VRTWNEIIPS 360
KGCLRVGGRC HPHVNGVFFN GIILGPDGNV LIPEMQSSLL QQHMELLVSS VIPLMHPLAD 420
PSTVFKNGDE AEDFVEVHLP DVHERISGVD LGLPNWGKYV LLSAGALTAL MLIIFLMTCW 480
RRVNRSEPTQ HNLRGTGREV SVTPQSGKII SSWESHKSGG ETRL 524

RVG according to SEQ ID NO: 2 excluding the N-terminal signal peptide: SEQ ID NO: 3

KFPIYTIPDK LGPWSPIDIH HLSCPNNLW EDEGCTNLSG FSYMELKVGY ISAIKMNGFT 60
CTGWTEAET YTNFVGYVTT TFKRKHFRPT PDACRAAYNW KMAGDPRYEE SLHNPYPDYH 120
WLRTVKTTKE SLVIISPSVA DLDPYDRSLH SPVFPGGNCS GVAVSSTYCS TNHDYTIWMP 180
ENPRLGMSCD IFTNSRGKRA SKGSETCGFV DERGLYKSLK GACKLKLCGV LGLRLMDGTW 240
VAMQTSNETK WCPPGQLVNL HDFRSDEIEH LVVEELVKKR EECLDALESI MTTKSVSFRR 300
LSHLRKLVPG FGKAYTIFNK TLMEADAHYK SVRTWNEIIP SKGCLRVGGR CHPHVNGVFF 360
NGIILGPDGN VLIPEMQSSL LQQHMELLVS SVIPLMHPLA DPSTVFKNGD EAEDFVEVHL 420
PDVHERISGV DLGLPNWGKY VLLSAGALTA LMLIIFLMTC WRRVNRSEPT QHNLRGTGRE 480
VSVTPQSGKI ISSWESHKSG GETRL 505

Full length VSVG (as described in Roche et al. 2006) including the N-terminal signal peptide: SEQ ID NO: 4

MKCLLYLAFL VNCKFTI VFPHNQKGNW KNVPSNYHYC PSSSDLNWHN DLIGTAIQVK 60
MPKSHKAIQA DGWMCHASKW VTTCDFRWYG PKYITQSIRS FTPSVEQCKE SIEQTKQGTW 120
LNPGFPPQSC GYATVTDAEA VIVQVTPHHV LVDEYTGEWV DSQFINGKCS NYICPTVHNS 180
TTWHSDYKVK GLCDSNLISM DITFFSEDGE LSSLGKEGTG FRSNYFAYET GGKACKMQYC 240
KHWGVRLPSG VWFEMADKDL FAAARFPECP EGSSISAPSQ TSVDVSLIQD VERILDYSLC 300
QETWSKIRAG LPISPVDLSY LAPKNPGTGP AFTIINGTLK YFETRYIRVD IAAPILSRMV 360
GMISGTTTER ELWDDWAPYE DVEIGPNGVL RTSSGYKFPL YMIGHGMLDS DLHLSSKAQV 420
FEHPHIQDAA SQLPDDESLF FGDTGLSKNP IELVEGWFSS WKSSIASFFF IIGLIIGLFL 480
VLRVGIHLCI KLKHTKKRQI YTDIEMNRLG K 511

Full length VSVG (as described in Roche et al. 2006) excluding the N-terminal signal peptide: SEQ ID NO: 5

KFTIVFPHNQ KGNWKNVPSN YHYCPSSSDL NWHNDLIGTA IQVKMPKSHK AIQADGWMCH 60
ASKWVTTCDF RWYGPKYITQ SIRSFTPSVE QCKESIEQTK QGTWLNPGFP PQSCGYATVT 120
DAEAVIVQVT PHHVLVDEYT GEWVDSQFIN GKCSNYICPT VHNSTTWHSD YKVKGLCDSN 180
LISMDITFFS EDGELSSLGK EGTGFRSNYF AYETGGKACK MQYCKHWGVR LPSGVWFEMA 240
DKDLFAAARF PECPEGSSIS APSQTSVDVS LIQDVERILD YSLCQETWSK IRAGLPISPV 300
DLSYLAPKNP GTGPAFTIIN GTLKYFETRY IRVDIAAPIL SRMVGMISGT TTERELWDDW 360
APYEDVEIGP NGVLRTSSGY KFPLYMIGHG MLDSDLHLSS KAQVFEHPHI QDAASQLPDD 420
ESLFFGDTGL SKNPIELVEG WFSSWKSSIA SFFFIIGLII GLFLVLRVGI HLCIKLKHTK 480
KRQIYTDIEM NRLGK 495

Full length EBV gB (Uniprot P03188) including the N-terminal signal peptide: SEQ ID NO: 6

MTRRRVLSW VLLAALACRL GAQTPEQPAP PATTVQPTAT RQQTSFPFRV CELSSHGDLF 60
RFSSDIQCPS FGTRENHTEG LLMVFKDNII PYSFKVRSYT KIVTNILIYN GWYADSVTNR 120
HEEKFSVDSY ETDQMDTIYQ CYNAVKMTKD GLTRVYVDRD GVNITVNLKP TGGLANGVRR 180
YASQTELYDA PGWLIWTYRT RTTVNCLITD MMAKSNSPFD FFVTTTGQTV EMSPFYDGKN 240
KETFHERADS FHVRTNYKIV DYDNRGTNPQ GERRAFLDKG TYTLSWKLEN RTAYCPLQHW 300
QTFDSTIATE TGKSIHFVTD EGTSSFVTNT TVGIELPDAF KCIEEQVNKT MHEKYEAVQD 360
RYTKGQEAIT YFITSGGLLL AWLPLTPRSL ATVKNLTELT TPTSSPPSSP SPPAPSAARG 420
STPAAVLRRR RRDAGNATTP VPPTAPGKSL GTLNNPATVQ IQFAYDSLRR QINRMLGDLA 480
RAWCLEQKRQ NMVLRELTKI NPTTVMSSIY GKAVAAKRLG DVISVSQCVP VNQATVTLRK 540
SMRVPGSETM CYSRPLVSFS FINDTKTYEG QLGTDNEIFL TKKMTEVCQA TSQYYFQSGN 600
EIHVYNDYHH FKTIELDGIA TLQTFISLNT SLIENIDFAS LELYSRDEQR ASNVFDLEGI 660
FREYNFQAQN IAGLRKDLDN AVSNGRNQFV DGLGELMDSL GSVGQSITNL VSTVGGLFSS 720
LVSGFISFFK NPFGGMLILV LVAGVVILVI SLTRRTRQMS QQPVQMLYPG IDELAQQHAS 780
GEGPGINPIS KTELQAIMLA LHEQNQEQKR AAQRAAGPSV ASRALQAARD RFPGLRRRRY 840
HDPETAAALL GEAETEF 857

EBV gB expression construct including an exogenous N-terminal signal peptide: SEQ ID NO: 7

MDAMKRGLCC VLLLCGAVFV SPSASQTPEQ PAPPATTVQP TATRQQTSFP FRVCELSSHG 60
DLFRFSSDIQ CPSFGTRENH TEGLLMVFKD NIIPYSFKVR SYTKIVTNIL IYNGHRADSV 120
TNRHEEKFSV DSYETDQMDT IYQCYNAVKM TKDGLTRVYV DRDGVNITVN LKPTGGLANG 180
VRRYASQTEL YDAPGRVEAT YRTRTTVNCL ITDMMAKSNS PFDFFVTTTG QTVEMSPFYD 240
GKNKETFHER ADSFHVRTNY KIVDYDNRGT NPQGERRAFL DKGTYTLSWK LENRTAYCPL 300
QHWQTFDSTI ATETGKSIHF VTDEGTSSFV TNTTVGIELP DAFKCIEEQV NKTMHEKYEA 360
VQDRYTKGQE AITYFITSGG LLLAWLPLTP RSLATVKNLT ELTTPTSSPP SSPSPPAPSA 420
ARGSTPAAVL RRRRRDAGNA TTPVPPTAPG KSLGTLNNPA TVQIQFAYDS LRRQINRMLG 480
DLARAWCLEQ KRQNMVLREL TKINPTTVMS SIYGKAVAAK RLGDVISVSQ CVPVNQATVT 540
LRKSMRVPGS ETMCYSRPLV SFSFINDTKT YEGQLGTDNE IFLTKKMTEV CQATSQYYFQ 600
SGNEIHVYND YHHFKTIELD GIATLQTFIS LNTSLIENID FASLELYSRD EQRASNVFDL 660
EGIFREYNFQ AQNIAGLRKD LDNAVSNGGS GSGHHHHHHG LNDIFEAQKI EWHE 714

Full length human CMV gB (Uniprot P13201) including the N-terminal signal peptide: SEQ ID NO: 8

MESRIWCLW CVNLCIVCLG AAVSSSSTRG TSATHSHHSS HTTSAAHSRS GSVSQRVTSS 60
QTVSHGVNET IYNTTLKYGD WGVNTTKYP YRVCSMAQGT DLIRFERNIV CTSMKPINED 120
LDEGIMWYK RNIVAHTFKV RVYQKVLTFR RSYAYIHTTY LLGSNTEYVA PPMWEIHHIN 180
SHSQCYSSYS RVIAGTVFVA YHRDSYENKT MQLMPDDYSN THSTRYVTVK DQWHSRGSTW 240
LYRETCNLNC MVTITTARSK YPYHFFATST GDVVDISPFY NGTNRNASYF GENADKFFIF 300
PNYTIVSDFG RPNSALETHR LVAFLERADS VISWDIQDEK NVTCQLTFWE ASERTIRSEA 360
EDSYHFSSAK MTATFLSKKQ EVNMSDSALD CVRDEAINKL QQIFNTSYNQ TYEKYGNVSV 420
FETTGGLWF WQGIKQKSLV ELERLANRSS LNLTHNRTKR STDGNNATHL SNMESVHNLV 480
YAQLQFTYDT LRGYINRALA QIAEAWCVDQ RRTLEVFKEL SKINPSAILS AIYNKPIAAR 540
FMGDVLGLAS CVTINQTSVK VLRDMNVKES PGRCYSRPVV IFNFANSSYV QYGQLGEDNE 600
ILLGNHRTEE CQLPSLKIFI AGNSAYEYVD YLFKRMIDLS SISTVDSMIA LDIDPLENTD 660
FRVLELYSQK ELRSSNVFDL EEIMREFNSY KQRVKYVEDK VVDPLPPYLK GLDDLMSGLG 720
AAGKAVGVAI GAVGGAVASV VEGVATFLKN PFGAFTIILV AIAVVIIIYL IYTRQRRLCM 780
QPLQNLFPYL VSADGTTVTS GNTKDTSLQA PPSYEESVYN SGRKGPGPPS SDASTAAPPY 840
TNEQAYQMLL ALVRLDAEQR AQQNGTDSLD GQTGTQDKGQ KPNLLDRLRH RKNGYRHLKD 900
SDEEENV 907

CMV gB expression construct including the N-terminal signal peptide and affinity tag: SEQ ID NO: 9

MESRIWCLW CVNLCIVCLG AAVSSSSTRG TSATHSHHSS HTTSAAHSRS GSVSQRVTSS 60
QTVSHGVNET IYNTTLKYGD WGVNTTKYP YRVCSMAQGT DLIRFERNIV CTSMKPINED 120
LDEGIMWYK RNIVAHTFKV RVYQKVLTFR RSYAYHRTTY LLGSNTEYVA PPMWEIHHIN 180
SHSQCYSSYS RVIAGTVFVA YHRDSYENKT MQLMPDDYSN THSTRYVTVK DQWHSRGSTN 240
LTRETSNLNC MVTITTARSK YPYHFFATST GDVVDISPFY NGTNRNASYF GENADKFFIF 300
PNYTIVSDFG RPNSALETHR LVAFLERADS VISWDIQDEK NVTCQLTFWE ASERTIRSEA 360
EDSYHFSSAK MTATFLSKKQ EVNMSDSALD CVRDEAINKL QQIFNTSYNQ TYEKYGNVSV 420
FETTGGLWF WQGIKQKSLV ELERLANRSS LNLTHNSTKS STDGNNATHL SNMESVHNLV 480
YAQLQFTYDT LRGYINRALA QIAEAWCVDQ RRTLEVFKEL SKINPSAILS AIYNKPIAAR 540
FMGDVLGLAS CVTINQTSVK VLRDMNVKES PGRCYSRPVV IFNFANSSYV QYGQLGEDNE 600
ILLGNHRTEE CQLPSLKIFI AGNSAYEYVD YLFKRMIDLS SISTVDSMIA LDIDPLENTD 660
FRVLELYSQK ELRSSNVFDL EEIMREFNSY KQRVKYVEGS AWSHPQFEK 709

Full length HSV-1 gB (as described in Heldwein et al. 2006, Uniprot P06437) including the N-terminal signal peptide: SEQ ID NO: 10

MHQGAPSWGR RWFVVWALLG LTLGVLVASA APTSPGTPGV AAATQAANGG PATPAPPPLG 60
AAPTGDPKPK KNKKPKNPTP PRPAGDNATV AAGHATLREH LRDIKAENTD ANFYVCPPPT 120
GATWQFEQP RRCPTRPEGQ NYTEGIAVVF KENIAPYKFK ATMYYKDVTV SQVWFGHRYS 180
QFMGIFEDRA PVPFEEVIDK INAKGVCRST AKYVRNNLET TAFHRDDHET DMELKPANAA 240
TRTSRGWHTT DLKYNPSRVE AFHRYGTTVN CIVEEVDARS VYPYDEFVLA TGDFVYMSPF 300
YGYREGSHTE HTTYAADRFK QVDGFYARDL TTKARATAPT TRNLLTTPKF TVAWDWVPKR 360
PSVCTMTKWQ EVDEMLRSEY GGSFRFSSDA ISTTFTTNLT EYPLSRVDLG DCIGKDARDA 420
MDRIFARRYN ATHIKVGQPQ YYQANGGFLI AYQPLLSNTL AELYVREHLR EQSRKPPNPT 480
PPPPGASANA SVERIKTTSS IEFARLQFTY NHIQRHVNDM LGRVAIAWCE LQNHELTLWN 540
EARKLNPNAI ASVTVGRRVS ARMLGDVMAV STCVPVAADN VIVQNSMRIS SRPGACYSRP 600
LVSFRYEDQG PLVEGQLGEN NELRLTRDAI EPCTVGHRRY FTFGGGYVYF EEYAYSHQLS 660
RADITTVSTF IDLNITMLED HEFVPLEVYT RHEIKDSGLL DYTEVQRRNQ LHDLRFADID 720
TVIHADANAA MFAGLGAFFE GMGDLGRAVG KVVMGIVGGV VSAVSGVSSF MSNPFGALAV 780
GLLVLAGLAA AFFAFRYVMR LQSNPMKALY PLTTKELKNP TNPDASGEGE EGGDFDEAKL 840
AEAREMIRYM ALVSAMERTE HKAKKKGTSA LLSAKVTDMV MRKRRNTNYT QVPNKDGDAD 900
EDDL 904

RVG comprising the H270P mutation: SEQ ID NO: 11

KFPIYTIPDK LGPWSPIDIH HLSCPNNLW EDEGCTNLSG FSYMELKVGY ISAIKMNGFT 60
CTGWTEAET YTNFVGYVTT TFKRKHFRPT PDACRAAYNW KMAGDPRYEE SLHNPYPDYH 120
WLRTVKTTKE SLVIISPSVA DLDPYDRSLH SPVFPGGNCS GVAVSSTYCS TNHDYTIWMP 180
ENPRLGMSCD IFTNSRGKRA SKGSETCGFV DERGLYKSLK GACKLKLCGV LGLRLMDGTW 240
VAMQTSNETK WCPPGQLVNL HDFRSDEIEP LVVEELVKKR EECLDALESI MTTKSVSFRR 300
LSHLRKLVPG FGKAYTIFNK TLMEADAHYK SVRTWNEIIP SKGCLRVGGR CHPHVNGVFF 360
NGIILGPDGN VLIPEMQSSL LQQHMELLVS SVIPLMHPLA DPSTVFKNGD EAEDFVEVHL 420
PDVHERISGV DLGLPNWGKY VLLSAGALTA LMLIIFLMTC WRRVNRSEPT QHNLRGTGRE 480
VSVTPQSGKI ISSWESHKSG GETRL 505

RVG comprising the L271P mutation: SEQ ID NO: 12

KFPIYTIPDK LGPWSPIDIH HLSCPNNLW EDEGCTNLSG FSYMELKVGY ISAIKMNGFT 60
CTGWTEAET YTNFVGYVTT TFKRKHFRPT PDACRAAYNW KMAGDPRYEE SLHNPYPDYH 120
WLRTVKTTKE SLVIISPSVA DLDPYDRSLH SPVFPGGNCS GVAVSSTYCS TNHDYTIWMP 180
ENPRLGMSCD IFTNSRGKRA SKGSETCGFV DERGLYKSLK GACKLKLCGV LGLRLMDGTW 240
VAMQTSNETK WCPPGQLVNL HDFRSDEIEH PVVEELVKKR EECLDALESI MTTKSVSFRR 300
LSHLRKLVPG FGKAYTIFNK TLMEADAHYK SVRTWNEIIP SKGCLRVGGR CHPHVNGVFF 360
NGIILGPDGN VLIPEMQSSL LQQHMELLVS SVIPLMHPLA DPSTVFKNGD EAEDFVEVHL 420
PDVHERISGV DLGLPNWGKY VLLSAGALTA LMLIIFLMTC WRRVNRSEPT QHNLRGTGRE 480
VSVTPQSGKI ISSWESHKSG GETRL 505

RVG comprising the V272P mutation: SEQ ID NO: 13

KFPIYTIPDK LGPWSPIDIH HLSCPNNLW EDEGCTNLSG FSYMELKVGY ISAIKMNGFT 60
CTGWTEAET YTNFVGYVTT TFKRKHFRPT PDACRAAYNW KMAGDPRYEE SLHNPYPDYH 120
WLRTVKTTKE SLVIISPSVA DLDPYDRSLH SPVFPGGNCS GVAVSSTYCS TNHDYTIWMP 180
ENPRLGMSCD IFTNSRGKRA SKGSETCGFV DERGLYKSLK GACKLKLCGV LGLRLMDGTW 240
VAMQTSNETK WCPPGQLVNL HDFRSDEIEH LPVEELVKKR EECLDALESI MTTKSVSFRR 300
LSHLRKLVPG FGKAYTIFNK TLMEADAHYK SVRTWNEIIP SKGCLRVGGR CHPHVNGVFF 360
NGIILGPDGN VLIPEMQSSL LQQHMELLVS SVIPLMHPLA DPSTVFKNGD EAEDFVEVHL 420
PDVHERISGV DLGLPNWGKY VLLSAGALTA LMLIIFLMTC WRRVNRSEPT QHNLRGTGRE 480
VSVTPQSGKI ISSWESHKSG GETRL 505

RVG comprising the V273P mutation: SEQ ID NO: 14

KFPIYTIPDK LGPWSPIDIH HLSCPNNLW EDEGCTNLSG FSYMELKVGY ISAIKMNGFT 60
CTGWTEAET YTNFVGYVTT TFKRKHFRPT PDACRAAYNW KMAGDPRYEE SLHNPYPDYH 120
WLRTVKTTKE SLVIISPSVA DLDPYDRSLH SPVFPGGNCS GVAVSSTYCS TNHDYTIWMP 180
ENPRLGMSCD IFTNSRGKRA SKGSETCGFV DERGLYKSLK GACKLKLCGV LGLRLMDGTW 240
VAMQTSNETK WCPPGQLVNL HDFRSDEIEH LVPEELVKKR EECLDALESI MTTKSVSFRR 300
LSHLRKLVPG FGKAYTIFNK TLMEADAHYK SVRTWNEIIP SKGCLRVGGR CHPHVNGVFF 360
NGIILGPDGN VLIPEMQSSL LQQHMELLVS SVIPLMHPLA DPSTVFKNGD EAEDFVEVHL 420
PDVHERISGV DLGLPNWGKY VLLSAGALTA LMLIIFLMTC WRRVNRSEPT QHNLRGTGRE 480
VSVTPQSGKI ISSWESHKSG GETRL 505

VSVG comprising the L271P mutation: SEQ ID NO: 15

KFTIVFPHNQ KGNWKNVPSN YHYCPSSSDL NWHNDLIGTA IQVKMPKSHK AIQADGWMCH 60
ASKWVTTCDF RWYGPKYITQ SIRSFTPSVE QCKESIEQTK QGTWLNPGFP PQSCGYATVT 120
DAEAVIVQVT PHHVLVDEYT GEWVDSQFIN GKCSNYICPT VHNSTTWHSD YKVKGLCDSN 180
LISMDITFFS EDGELSSLGK EGTGFRSNYF AYETGGKACK MQYCKHWGVR LPSGVWFEMA 240
DKDLFAAARF PECPEGSSIS APSQTSVDVS PIQDVERILD YSLCQETWSK IRAGLPISPV 300
DLSYLAPKNP GTGPAFTIIN GTLKYFETRY IRVDIAAPIL SRMVGMISGT TTERELWDDW 360
APYEDVEIGP NGVLRTSSGY KFPLYMIGHG MLDSDLHLSS KAQVFEHPHI QDAASQLPDD 420
ESLFFGDTGL SKNPIELVEG WFSSWKSSIA SFFFIIGLII GLFLVLRVGI HLCIKLKHTK 480
KRQIYTDIEM NRLGK 495

VSVG comprising the I272P mutation: SEQ ID NO: 16

KFTIVFPHNQ KGNWKNVPSN YHYCPSSSDL NWHNDLIGTA IQVKMPKSHK AIQADGWMCH 60
ASKWVTTCDF RWYGPKYITQ SIRSFTPSVE QCKESIEQTK QGTWLNPGFP PQSCGYATVT 120
DAEAVIVQVT PHHVLVDEYT GEWVDSQFIN GKCSNYICPT VHNSTTWHSD YKVKGLCDSN 180
LISMDITFFS EDGELSSLGK EGTGFRSNYF AYETGGKACK MQYCKHWGVR LPSGVWFEMA 240
DKDLFAAARF PECPEGSSIS APSQTSVDVS LPQDVERILD YSLCQETWSK IRAGLPISPV 300
DLSYLAPKNP GTGPAFTIIN GTLKYFETRY IRVDIAAPIL SRMVGMISGT TTERELWDDW 360
APYEDVEIGP NGVLRTSSGY KFPLYMIGHG MLDSDLHLSS KAQVFEHPHI QDAASQLPDD 420
ESLFFGDTGL SKNPIELVEG WFSSWKSSIA SFFFIIGLII GLFLVLRVGI HLCIKLKHTK 480
KRQIYTDIEM NRLGK 495

VSVG comprising the Q273P mutation: SEQ ID NO: 17

KFTIVFPHNQ KGNWKNVPSN YHYCPSSSDL NWHNDLIGTA IQVKMPKSHK AIQADGWMCH 60
ASKWVTTCDF RWYGPKYITQ SIRSFTPSVE QCKESIEQTK QGTWLNPGFP PQSCGYATVT 120
DAEAVIVQVT PHHVLVDEYT GEWVDSQFIN GKCSNYICPT VHNSTTWHSD YKVKGLCDSN 180
LISMDITFFS EDGELSSLGK EGTGFRSNYF AYETGGKACK MQYCKHWGVR LPSGVWFEMA 240
DKDLFAAARF PECPEGSSIS APSQTSVDVS LIPDVERILD YSLCQETWSK IRAGLPISPV 300
DLSYLAPKNP GTGPAFTIIN GTLKYFETRY IRVDIAAPIL SRMVGMISGT TTERELWDDW 360
APYEDVEIGP NGVLRTSSGY KFPLYMIGHG MLDSDLHLSS KAQVFEHPHI QDAASQLPDD 420
ESLFFGDTGL SKNPIELVEG WFSSWKSSIA SFFFIIGLII GLFLVLRVGI HLCIKLKHTK 480
KRQIYTDIEM NRLGK 495

VSVG comprising the D274P mutation: SEQ ID NO: 18

KFTIVFPHNQ KGNWKNVPSN YHYCPSSSDL NWHNDLIGTA IQVKMPKSHK AIQADGWMCH 60
ASKWVTTCDF RWYGPKYITQ SIRSFTPSVE QCKESIEQTK QGTWLNPGFP PQSCGYATVT 120
DAEAVIVQVT PHHVLVDEYT GEWVDSQFIN GKCSNYICPT VHNSTTWHSD YKVKGLCDSN 180
LISMDITFFS EDGELSSLGK EGTGFRSNYF AYETGGKACK MQYCKHWGVR LPSGVWFEMA 240
DKDLFAAARF PECPEGSSIS APSQTSVDVS LIQPVERILD YSLCQETWSK IRAGLPISPV 300
DLSYLAPKNP GTGPAFTIIN GTLKYFETRY IRVDIAAPIL SRMVGMISGT TTERELWDDW 360
APYEDVEIGP NGVLRTSSGY KFPLYMIGHG MLDSDLHLSS KAQVFEHPHI QDAASQLPDD 420
ESLFFGDTGL SKNPIELVEG WFSSWKSSIA SFFFIIGLII GLFLVLRVGI HLCIKLKHTK 480
KRQIYTDIEM NRLGK 495

EBV gB comprising the Q471P mutation: SEQ ID NO: 19

MDAMKRGLCC VLLLCGAVFV SPSASQTPEQ PAPPATTVQP TATRQQTSFP FRVCELSSHG 60
DLFRFSSDIQ CPSFGTRENH TEGLLMVFKD NIIPYSFKVR SYTKIVTNIL IYNGHRADSV 120
TNRHEEKFSV DSYETDQMDT IYQCYNAVKM TKDGLTRVYV DRDGVNITVN LKPTGGLANG 180
VRRYASQTEL YDAPGRVEAT YRTRTTVNCL ITDMMAKSNS PFDFFVTTTG QTVEMSPFYD 240
GKNKETFHER ADSFHVRTNY KIVDYDNRGT NPQGERRAFL DKGTYTLSWK LENRTAYCPL 300
QHWQTFDSTI ATETGKSIHF VTDEGTSSFV TNTTVGIELP DAFKCIEEQV NKTMHEKYEA 360
VQDRYTKGQE AITYFITSGG LLLAWLPLTP RSLATVKNLT ELTTPTSSPP SSPSPPAPSA 420
ARGSTPAAVL RRRRRDAGNA TTPVPPTAPG KSLGTLNNPA TVQIQFAYDS LRRPINRMLG 480
DLARAWCLEQ KRQNMVLREL TKINPTTVMS SIYGKAVAAK RLGDVISVSQ CVPVNQATVT 540
LRKSMRVPGS ETMCYSRPLV SFSFINDTKT YEGQLGTDNE IFLTKKMTEV CQATSQYYFQ 600
SGNEIHVYND YHHFKTIELD GIATLQTFIS LNTSLIENID FASLELYSRD EQRASNVFDL 660
EGIFREYNFQ AQNIAGLRKD LDNAVSNGGS GSGHHHHHHG LNDIFEAQKI EWHE 714

EBV gB comprising the I472P mutation: SEQ ID NO: 20

MDAMKRGLCC VLLLCGAVFV SPSASQTPEQ PAPPATTVQP TATRQQTSFP FRVCELSSHG 60
DLFRFSSDIQ CPSFGTRENH TEGLLMVFKD NIIPYSFKVR SYTKIVTNIL IYNGHRADSV 120
TNRHEEKFSV DSYETDQMDT IYQCYNAVKM TKDGLTRVYV DRDGVNITVN LKPTGGLANG 180
VRRYASQTEL YDAPGRVEAT YRTRTTVNCL ITDMMAKSNS PFDFFVTTTG QTVEMSPFYD 240
GKNKETFHER ADSFHVRTNY KIVDYDNRGT NPQGERRAFL DKGTYTLSWK LENRTAYCPL 300
QHWQTFDSTI ATETGKSIHF VTDEGTSSFV TNTTVGIELP DAFKCIEEQV NKTMHEKYEA 360
VQDRYTKGQE AITYFITSGG LLLAWLPLTP RSLATVKNLT ELTTPTSSPP SSPSPPAPSA 420
ARGSTPAAVL RRRRRDAGNA TTPVPPTAPG KSLGTLNNPA TVQIQFAYDS LRRQPNRMLG 480
DLARAWCLEQ KRQNMVLREL TKINPTTVMS SIYGKAVAAK RLGDVISVSQ CVPVNQATVT 540
LRKSMRVPGS ETMCYSRPLV SFSFINDTKT YEGQLGTDNE IFLTKKMTEV CQATSQYYFQ 600
SGNEIHVYND YHHFKTIELD GIATLQTFIS LNTSLIENID FASLELYSRD EQRASNVFDL 660
EGIFREYNFQ AQNIAGLRKD LDNAVSNGGS GSGHHHHHHG LNDIFEAQKI EWHE 714

EBV gB comprising the N473P mutation: SEQ ID NO: 21

MDAMKRGLCC VLLLCGAVFV SPSASQTPEQ PAPPATTVQP TATRQQTSFP FRVCELSSHG 60
DLFRFSSDIQ CPSFGTRENH TEGLLMVFKD NIIPYSFKVR SYTKIVTNIL IYNGHRADSV 120
TNRHEEKFSV DSYETDQMDT IYQCYNAVKM TKDGLTRVYV DRDGVNITVN LKPTGGLANG 180
VRRYASQTEL YDAPGRVEAT YRTRTTVNCL ITDMMAKSNS PFDFFVTTTG QTVEMSPFYD 240
GKNKETFHER ADSFHVRTNY KIVDYDNRGT NPQGERRAFL DKGTYTLSWK LENRTAYCPL 300
QHWQTFDSTI ATETGKSIHF VTDEGTSSFV TNTTVGIELP DAFKCIEEQV NKTMHEKYEA 360
VQDRYTKGQE AITYFITSGG LLLAWLPLTP RSLATVKNLT ELTTPTSSPP SSPSPPAPSA 420
ARGSTPAAVL RRRRRDAGNA TTPVPPTAPG KSLGTLNNPA TVQIQFAYDS LRRQIPRMLG 480
DLARAWCLEQ KRQNMVLREL TKINPTTVMS SIYGKAVAAK RLGDVISVSQ CVPVNQATVT 540
LRKSMRVPGS ETMCYSRPLV SFSFINDTKT YEGQLGTDNE IFLTKKMTEV CQATSQYYFQ 600
SGNEIHVYND YHHFKTIELD GIATLQTFIS LNTSLIENID FASLELYSRD EQRASNVFDL 660
EGIFREYNFQ AQNIAGLRKD LDNAVSNGGS GSGHHHHHHG LNDIFEAQKI EWHE 714

EBV gB comprising the R474P mutation: SEQ ID NO: 22

MDAMKRGLCC VLLLCGAVFV SPSASQTPEQ PAPPATTVQP TATRQQTSFP FRVCELSSHG 60
DLFRFSSDIQ CPSFGTRENH TEGLLMVFKD NIIPYSFKVR SYTKIVTNIL IYNGHRADSV 120
TNRHEEKFSV DSYETDQMDT IYQCYNAVKM TKDGLTRVYV DRDGVNITVN LKPTGGLANG 180
VRRYASQTEL YDAPGRVEAT YRTRTTVNCL ITDMMAKSNS PFDFFVTTTG QTVEMSPFYD 240
GKNKETFHER ADSFHVRTNY KIVDYDNRGT NPQGERRAFL DKGTYTLSWK LENRTAYCPL 300
QHWQTFDSTI ATETGKSIHF VTDEGTSSFV TNTTVGIELP DAFKCIEEQV NKTMHEKYEA 360
VQDRYTKGQE AITYFITSGG LLLAWLPLTP RSLATVKNLT ELTTPTSSPP SSPSPPAPSA 420
ARGSTPAAVL RRRRRDAGNA TTPVPPTAPG KSLGTLNNPA TVQIQFAYDS LRRQINPMLG 480
DLARAWCLEQ KRQNMVLREL TKINPTTVMS SIYGKAVAAK RLGDVISVSQ CVPVNQATVT 540
LRKSMRVPGS ETMCYSRPLV SFSFINDTKT YEGQLGTDNE IFLTKKMTEV CQATSQYYFQ 600
SGNEIHVYND YHHFKTIELD GIATLQTFIS LNTSLIENID FASLELYSRD EQRASNVFDL 660
EGIFREYNFQ AQNIAGLRKD LDNAVSNGGS GSGHHHHHHG LNDIFEAQKI EWHE 714

CMV gB comprising the Y494P mutation: SEQ ID NO: 23

LDEGIMWYK RNIVAHTFKV RVYQKVLTFR RSYAYHRTTY LLGSNTEYVA PPMWEIHHIN 180
SHSQCYSSYS RVIAGTVFVA YHRDSYENKT MQLMPDDYSN THSTRYVTVK DQWHSRGSTN 240
LTRETSNLNC MVTITTARSK YPYHFFATST GDVVDISPFY NGTNRNASYF GENADKFFIF 300
PNYTIVSDFG RPNSALETHR LVAFLERADS VISWDIQDEK NVTCQLTFWE ASERTIRSEA 360
EDSYHFSSAK MTATFLSKKQ EVNMSDSALD CVRDEAINKL QQIFNTSYNQ TYEKYGNVSV 420
FETTGGLWF WQGIKQKSLV ELERLANRSS LNLTHNSTKS STDGNNATHL SNMESVHNLV 480
YAQLQFTYDT LRGPINRALA QIAEAWCVDQ RRTLEVFKEL SKINPSAILS AIYNKPIAAR 540
FMGDVLGLAS CVTINQTSVK VLRDMNVKES PGRCYSRPVV IFNFANSSYV QYGQLGEDNE 600
ILLGNHRTEE CQLPSLKIFI AGNSAYEYVD YLFKRMIDLS SISTVDSMIA LDIDPLENTD 660
FRVLELYSQK ELRSSNVFDL EEIMREFNSY KQRVKYVEGS AWSHPQFEK 709

CMV gB comprising the I495P mutation: SEQ ID NO: 24

MESRIWCLW CVNLCIVCLG AAVSSSSTRG TSATHSHHSS HTTSAAHSRS GSVSQRVTSS 60
QTVSHGVNET IYNTTLKYGD WGVNTTKYP YRVCSMAQGT DLIRFERNIV CTSMKPINED 120
LDEGIMWYK RNIVAHTFKV RVYQKVLTFR RSYAYHRTTY LLGSNTEYVA PPMWEIHHIN 180
SHSQCYSSYS RVIAGTVFVA YHRDSYENKT MQLMPDDYSN THSTRYVTVK DQWHSRGSTN 240
LTRETSNLNC MVTITTARSK YPYHFFATST GDVVDISPFY NGTNRNASYF GENADKFFIF 300
PNYTIVSDFG RPNSALETHR LVAFLERADS VISWDIQDEK NVTCQLTFWE ASERTIRSEA 360
EDSYHFSSAK MTATFLSKKQ EVNMSDSALD CVRDEAINKL QQIFNTSYNQ TYEKYGNVSV 420
FETTGGLWF WQGIKQKSLV ELERLANRSS LNLTHNSTKS STDGNNATHL SNMESVHNLV 480
YAQLQFTYDT LRGYPNRALA QIAEAWCVDQ RRTLEVFKEL SKINPSAILS AIYNKPIAAR 540
FMGDVLGLAS CVTINQTSVK VLRDMNVKES PGRCYSRPVV IFNFANSSYV QYGQLGEDNE 600
ILLGNHRTEE CQLPSLKIFI AGNSAYEYVD YLFKRMIDLS SISTVDSMIA LDIDPLENTD 660
FRVLELYSQK ELRSSNVFDL EEIMREFNSY KQRVKYVEGS AWSHPQFEK 709

CMV gB comprising the N496P mutation: SEQ ID NO: 25

MESRIWCLVV CVNLCIVCLG AAVSSSSTRG TSATHSHHSS HTTSAAHSRS GSVSQRVTSS 60
QTVSHGVNET IYNTTLKYGD WGVNTTKYP YRVCSMAQGT DLIRFERNIV CTSMKPINED 120
LDEGIMWYK RNIVAHTFKV RVYQKVLTFR RSYAYHRTTY LLGSNTEYVA PPMWEIHHIN 180
SHSQCYSSYS RVIAGTVFVA YHRDSYENKT MQLMPDDYSN THSTRYVTVK DQWHSRGSTN 240
LTRETSNLNC MVTITTARSK YPYHFFATST GDVVDISPFY NGTNRNASYF GENADKFFIF 300
PNYTIVSDFG RPNSALETHR LVAFLERADS VISWDIQDEK NVTCQLTFWE ASERTIRSEA 360
EDSYHFSSAK MTATFLSKKQ EVNMSDSALD CVRDEAINKL QQIFNTSYNQ TYEKYGNVSV 420
FETTGGLWF WQGIKQKSLV ELERLANRSS LNLTHNSTKS STDGNNATHL SNMESVHNLV 480
YAQLQFTYDT LRGYIPRALA QIAEAWCVDQ RRTLEVFKEL SKINPSAILS AIYNKPIAAR 540
FMGDVLGLAS CVTINQTSVK VLRDMNVKES PGRCYSRPVV IFNFANSSYV QYGQLGEDNE 600
ILLGNHRTEE CQLPSLKIFI AGNSAYEYVD YLFKRMIDLS SISTVDSMIA LDIDPLENTD 660
FRVLELYSQK ELRSSNVFDL EEIMREFNSY KQRVKYVEGS AWSHPQFEK 709

CMV gB comprising the R497P mutation: SEQ ID NO: 26

MESRIWCLVV CVNLCIVCLG AAVSSSSTRG TSATHSHHSS HTTSAAHSRS GSVSQRVTSS 60
QTVSHGVNET IYNTTLKYGD WGVNTTKYP YRVCSMAQGT DLIRFERNIV CTSMKPINED 120
LDEGIMWYK RNIVAHTFKV RVYQKVLTFR RSYAYHRTTY LLGSNTEYVA PPMWEIHHIN 180
SHSQCYSSYS RVIAGTVFVA YHRDSYENKT MQLMPDDYSN THSTRYVTVK DQWHSRGSTN 240
LTRETSNLNC MVTITTARSK YPYHFFATST GDVVDISPFY NGTNRNASYF GENADKFFIF 300
PNYTIVSDFG RPNSALETHR LVAFLERADS VISWDIQDEK NVTCQLTFWE ASERTIRSEA 360
EDSYHFSSAK MTATFLSKKQ EVNMSDSALD CVRDEAINKL QQIFNTSYNQ TYEKYGNVSV 420
FETTGGLWF WQGIKQKSLV ELERLANRSS LNLTHNSTKS STDGNNATHL SNMESVHNLV 480
YAQLQFTYDT LRGYINPALA QIAEAWCVDQ RRTLEVFKEL SKINPSAILS AIYNKPIAAR 540
FMGDVLGLAS CVTINQTSVK VLRDMNVKES PGRCYSRPVV IFNFANSSYV QYGQLGEDNE 600
ILLGNHRTEE CQLPSLKIFI AGNSAYEYVD YLFKRMIDLS SISTVDSMIA LDIDPLENTD 660
FRVLELYSQK ELRSSNVFDL EEIMREFNSY KQRVKYVEGS AWSHPQFEK 709

HSV-1 gB comprising the H516P mutation: SEQ ID NO: 27

MHQGAPSWGR RWFVVWALLG LTLGVLVASA APTSPGTPGV AAATQAANGG PATPAPPPLG 60
AAPTGDPKPK KNKKPKNPTP PRPAGDNATV AAGHATLREH LRDIKAENTD ANFYVCPPPT 120
GATWQFEQP RRCPTRPEGQ NYTEGIAVVF KENIAPYKFK ATMYYKDVTV SQVWFGHRYS 180
QFMGIFEDRA PVPFEEVIDK INAKGVCRST AKYVRNNLET TAFHRDDHET DMELKPANAA 240
TRTSRGWHTT DLKYNPSRVE AFHRYGTTVN CIVEEVDARS VYPYDEFVLA TGDFVYMSPF 300
YGYREGSHTE HTTYAADRFK QVDGFYARDL TTKARATAPT TRNLLTTPKF TVAWDWVPKR 360
PSVCTMTKWQ EVDEMLRSEY GGSFRFSSDA ISTTFTTNLT EYPLSRVDLG DCIGKDARDA 420
MDRIFARRYN ATHIKVGQPQ YYQANGGFLI AYQPLLSNTL AELYVREHLR EQSRKPPNPT 480
PPPPGASANA SVERIKTTSS IEFARLQFTY NHIQRPVNDM LGRVAIAWCE LQNHELTLWN 540
EARKLNPNAI ASVTVGRRVS ARMLGDVMAV STCVPVAADN VIVQNSMRIS SRPGACYSRP 600
LVSFRYEDQG PLVEGQLGEN NELRLTRDAI EPCTVGHRRY FTFGGGYVYF EEYAYSHQLS 660
RADITTVSTF IDLNITMLED HEFVPLEVYT RHEIKDSGLL DYTEVQRRNQ LHDLRFADID 720
TVIHADANAA MFAGLGAFFE GMGDLGRAVG KVVMGIVGGV VSAVSGVSSF MSNPFGALAV 780
GLLVLAGLAA AFFAFRYVMR LQSNPMKALY PLTTKELKNP TNPDASGEGE EGGDFDEAKL 840
AEAREMIRYM ALVSAMERTE HKAKKKGTSA LLSAKVTDMV MRKRRNTNYT QVPNKDGDAD 900
EDDL 904

HSV-1 gB comprising the V517P mutation: SEQ ID NO: 28

MHQGAPSWGR RWFVVWALLG LTLGVLVASA APTSPGTPGV AAATQAANGG PATPAPPPLG 60
AAPTGDPKPK KNKKPKNPTP PRPAGDNATV AAGHATLREH LRDIKAENTD ANFYVCPPPT 120
GATWQFEQP RRCPTRPEGQ NYTEGIAVVF KENIAPYKFK ATMYYKDVTV SQVWFGHRYS 180
QFMGIFEDRA PVPFEEVIDK INAKGVCRST AKYVRNNLET TAFHRDDHET DMELKPANAA 240
TRTSRGWHTT DLKYNPSRVE AFHRYGTTVN CIVEEVDARS VYPYDEFVLA TGDFVYMSPF 300
YGYREGSHTE HTTYAADRFK QVDGFYARDL TTKARATAPT TRNLLTTPKF TVAWDWVPKR 360
PSVCTMTKWQ EVDEMLRSEY GGSFRFSSDA ISTTFTTNLT EYPLSRVDLG DCIGKDARDA 420
MDRIFARRYN ATHIKVGQPQ YYQANGGFLI AYQPLLSNTL AELYVREHLR EQSRKPPNPT 480
PPPPGASANA SVERIKTTSS IEFARLQFTY NHIQRHPNDM LGRVAIAWCE LQNHELTLWN 540
EARKLNPNAI ASVTVGRRVS ARMLGDVMAV STCVPVAADN VIVQNSMRIS SRPGACYSRP 600
LVSFRYEDQG PLVEGQLGEN NELRLTRDAI EPCTVGHRRY FTFGGGYVYF EEYAYSHQLS 660
RADITTVSTF IDLNITMLED HEFVPLEVYT RHEIKDSGLL DYTEVQRRNQ LHDLRFADID 720
TVIHADANAA MFAGLGAFFE GMGDLGRAVG KVVMGIVGGV VSAVSGVSSF MSNPFGALAV 780
GLLVLAGLAA AFFAFRYVMR LQSNPMKALY PLTTKELKNP TNPDASGEGE EGGDFDEAKL 840
AEAREMIRYM ALVSAMERTE HKAKKKGTSA LLSAKVTDMV MRKRRNTNYT QVPNKDGDAD 900
EDDL 904

HSV-1 gB comprising the N518P mutation: SEQ ID NO: 29

MHQGAPSWGR RWFVVWALLG LTLGVLVASA APTSPGTPGV AAATQAANGG PATPAPPPLG 60
AAPTGDPKPK KNKKPKNPTP PRPAGDNATV AAGHATLREH LRDIKAENTD ANFYVCPPPT 120
GATWQFEQP RRCPTRPEGQ NYTEGIAVVF KENIAPYKFK ATMYYKDVTV SQVWFGHRYS 180
QFMGIFEDRA PVPFEEVIDK INAKGVCRST AKYVRNNLET TAFHRDDHET DMELKPANAA 240
TRTSRGWHTT DLKYNPSRVE AFHRYGTTVN CIVEEVDARS VYPYDEFVLA TGDFVYMSPF 300
YGYREGSHTE HTTYAADRFK QVDGFYARDL TTKARATAPT TRNLLTTPKF TVAWDWVPKR 360
PSVCTMTKWQ EVDEMLRSEY GGSFRFSSDA ISTTFTTNLT EYPLSRVDLG DCIGKDARDA 420
MDRIFARRYN ATHIKVGQPQ YYQANGGFLI AYQPLLSNTL AELYVREHLR EQSRKPPNPT 480
PPPPGASANA SVERIKTTSS IEFARLQFTY NHIQRHVPDM LGRVAIAWCE LQNHELTLWN 540
EARKLNPNAI ASVTVGRRVS ARMLGDVMAV STCVPVAADN VIVQNSMRIS SRPGACYSRP 600
LVSFRYEDQG PLVEGQLGEN NELRLTRDAI EPCTVGHRRY FTFGGGYVYF EEYAYSHQLS 660
RADITTVSTF IDLNITMLED HEFVPLEVYT RHEIKDSGLL DYTEVQRRNQ LHDLRFADID 720
TVIHADANAA MFAGLGAFFE GMGDLGRAVG KVVMGIVGGV VSAVSGVSSF MSNPFGALAV 780
GLLVLAGLAA AFFAFRYVMR LQSNPMKALY PLTTKELKNP TNPDASGEGE EGGDFDEAKL 840
AEAREMIRYM ALVSAMERTE HKAKKKGTSA LLSAKVTDMV MRKRRNTNYT QVPNKDGDAD 900
EDDL 904

HSV-1 gB comprising the D519P mutation: SEQ ID NO: 30

MHQGAPSWGR RWFVVWALLG LTLGVLVASA APTSPGTPGV AAATQAANGG PATPAPPPLG 60
AAPTGDPKPK KNKKPKNPTP PRPAGDNATV AAGHATLREH LRDIKAENTD ANFYVCPPPT 120
GATWQFEQP RRCPTRPEGQ NYTEGIAVVF KENIAPYKFK ATMYYKDVTV SQVWFGHRYS 180
QFMGIFEDRA PVPFEEVIDK INAKGVCRST AKYVRNNLET TAFHRDDHET DMELKPANAA 240
TRTSRGWHTT DLKYNPSRVE AFHRYGTTVN CIVEEVDARS VYPYDEFVLA TGDFVYMSPF 300
YGYREGSHTE HTTYAADRFK QVDGFYARDL TTKARATAPT TRNLLTTPKF TVAWDWVPKR 360
PSVCTMTKWQ EVDEMLRSEY GGSFRFSSDA ISTTFTTNLT EYPLSRVDLG DCIGKDARDA 420
MDRIFARRYN ATHIKVGQPQ YYQANGGFLI AYQPLLSNTL AELYVREHLR EQSRKPPNPT 480
PPPPGASANA SVERIKTTSS IEFARLQFTY NHIQRHVNPM LGRVAIAWCE LQNHELTLWN 540
EARKLNPNAI ASVTVGRRVS ARMLGDVMAV STCVPVAADN VIVQNSMRIS SRPGACYSRP 600
LVSFRYEDQG PLVEGQLGEN NELRLTRDAI EPCTVGHRRY FTFGGGYVYF EEYAYSHQLS 660
RADITTVSTF IDLNITMLED HEFVPLEVYT RHEIKDSGLL DYTEVQRRNQ LHDLRFADID 720
TVIHADANAA MFAGLGAFFE GMGDLGRAVG KVVMGIVGGV VSAVSGVSSF MSNPFGALAV 780
GLLVLAGLAA AFFAFRYVMR LQSNPMKALY PLTTKELKNP TNPDASGEGE EGGDFDEAKL 840
AEAREMIRYM ALVSAMERTE HKAKKKGTSA LLSAKVTDMV MRKRRNTNYT QVPNKDGDAD 900
EDDL 904

Full-length wild-type RVG (SAD B19 strain) (Uniprot P16288): SEQ ID NO: 31

MVPQALLFVP LLVFPLCFGK FPIYTIPDKL GPWSPIDIHH LSCPNNLWE DEGCTNLSGF 60
SYMELKVGYI LAIKVNGFTC TGWTEAETY TNFVGYVTTT FKRKHFRPTP DACRAAYNWK 120
MAGDPRYEES LHNPYPDYRW LRTVKTTKES LVIISPSVAD LDPYDRSLHS RVFPSGKCSG 180
VAVSSTYCST NHDYTIWMPE NPRLGMSCDI FTNSRGKRAS KGSETCGFVD ERGLYKSLKG 240
ACKLKLCGVL GLRLMDGTWV SMQTSNETKW CPPDKLVNLH DFRSDEIEHL WEELVRKRE 300
ECLDALESIM TTKSVSFRRL SHLRKLVPGF GKAYTIFNKT LMEADAHYKS VRTWNEILPS 360
KGCLRVGGRC HPHVNGVFFN GIILGPDGNV LIPEMQSSLL QQHMELLESS VIPLVHPLAD 420
PSTVFKDGDE AEDFVEVHLP DVHNQVSGVD LGLPNWGKYV LLSAGALTAL MLIIFLMTCC 480
RRVNRSEPTQ HNLRGTGREV SVTPQSGKII SSWESHKSGG ETRL 524

Full length HSV-2 gB (Uniprot P06763) including the N-terminal signal peptide: SEQ ID NO: 32

MRGGGLICAL WGALVAAVA SAAPAAPAAP RASGGVAATV AANGGPASRP PPVPSPATTK 60
ARKRKTKKPP KRPEATPPPD ANATVAAGHA TLRAHLREIK VENADAQFYV CPPPTGATW 120
QFEQPRRCPT RPEGQNYTEG IAVVFKENIA PYKFKATMYY KDVTVSQVWF GHRYSQFMGI 180
FEDRAPVPFE EVIDKINAKG VCRSTAKYVR NNMETTAFHR DDHETDMELK PAKVATRTSR 240
GWHTTDLKYN PSRVEAFHRY GTTVNCIVEE VDARSVYPYD EFVLATGDFV YMSPFYGYRE 300
GSHTEHTSYA ADRFKQVDGF YARDLTTKAR ATSPTTRNLL TTPKFTVAWD WVPKRPAVCT 360
MTKWQEVDEM LRAEYGGSFR FSSDAISTTF TTNLTQYSLS RVDLGDCIGR DAREAIDRMF 420
ARKYNATHIK VGQPQYYLAT GGFLIAYQPL LSNTLAELYV REYMREQDRK PRNATPAPLR 480
EAPSANASVE RIKTTSSIEF ARLQFTYNHI QRHVNDMLGR IAVAWCELQN HELTLWNEAR 540
KLNPNAIASA TVGRRVSARM LGDVMAVSTC VPVAPDNVIV QNSMRVSSRP GTCYSRPLVS 600
FRYEDQGPLI EGQLGENNEL RLTRDALEPC TVGHRRYFIF GGGYVYFEEY AYSHQLSRAD 660
VTTVSTFIDL NITMLEDHEF VPLEVYTRHE IKDSGLLDYT EVQRRNQLHD LRFADIDTVI 720
RADANAAMFA GLCAFFEGMG DLGRAVGKW MGVVGGVVSA VSGVSSFMSN PFGALAVGLL 780
VLAGLVAAFF AFRYVLQLQR NPMKALYPLT TKELKTSDPG GVGGEGEEGA EGGGFDEAKL 840
AEAREMIRYM ALVSAMERTE HKARKKGTSA LLSSKVTNMV LRKRNKARYS PLHNEDEAGD 900
EDEL 904

The present invention will now be described with reference to the following non-limiting Examples.

### EXAMPLES

### Example 1: Design, Generation and Analysis of Mutant RVG Proteins

Models of RVG in pre-fusion and post-fusion form were prepared by an alignment of the query sequence (wildtype RVG sequence as defined by SEQ ID NO: 31) with possible templates using the LOMETS server (https://zhanglab.ccmb.med.umich.edu/LOMETS/), followed by submission of the alignments and template (PDB ID: 5I2S) to the homology modelling software RosettaCM22,

A set of candidate mutants were designed using the following strategies:
(i) Naturally-occurring mutations previously reported to confer resistance to acid-induced neutralisation (RAIN) in laboratory-selected rabies virus strains.
(ii) Mutations in the region of RVG aligning with the bend between helices F1 and F2 in VSV G, designed to hinder the formation of the extended helix F in the post-fusion conformation.
(iii) Mutations designed to replace histidine residues in locations which may be buried in the pre-fusion form but surface-exposed in the post-fusion form (protonation of which may result in the switch to the low pH form) with residues which would remain non-polar or hydrophobic (alanine and leucine).

Mutants were initially designed on the background of a construct comprising the wild-type Pasteur strain ectodomain and the wild-type SADB19 intravirion domain (this chimera is defined by SEQ ID NO: 2), fused at the C-terminus to green fluorescent protein. Additional experiments were performed using constructs lacking the GFP fusion (i.e. untagged full-length RVG).

Protein coding sequences were synthesized and cloned into the pTT5 transient mammalian expression plasmid and transiently transfected into Expi293 cells using Expifectamine (both from ThermoFisher). Cells were collected for analysis of expression after 2-4 days. Effective transfection was confirmed by measuring the expression of luciferase from a co-transfected plasmid.

Levels of pre-fusion conformation RVG were measured by FACS on transfected cell surface and by ELISA in detergent-solubilised cell lysate by flow cytometry using monoclonal antibody 1112-1, which is known to neutralise rabies virus, to mediate protection against rabies challenge upon passive transfer into mice, and to bind to the site II epitope which is present only in the neutral (pre-fusion) conformation. For each sample, flow cytometry was performed after incubation of transfected cells with 1112-1 under neutral pH conditions (pH 7.4) and under acidic conditions (pH 5.8). Allophycocyanin-(APC) labelled anti-mouse-immunoglobulin was used as a secondary / detection reagent. Data was acquired on an LSR-II cytometer (BD Biosciences). Median APC fluorescence intensity (MFI) in samples stained at pH 7.4 was used as an index of RVG expression. For a given construct, the ratio of APC MFI in the sample stained at pH 5.8 to that in the sample stained at pH 7.4 was used as an index of pre-fusion stability. The results of the FACS analysis for GFP-tagged mutants and the un-tagged mutants are provided in Tables 1 and 2 respectively.

TABLE 1

FACS analysis of RVG-GFP tagged mutants

| Mutation | Group/ rationale | MFI at pH 7.4 | MFI at pH 5.8 | Ratio MFI 5.8/7.4 |
|---|---|---|---|---|
| WT | N/A | 1244 | 392 | 0.3 |
| M44V | RAIN mutation | 395 | 156 | 0.4 |
| M44I | | 275 | 130 | 0.5 |
| H270P | Prevention of F-helix formation | 3823 | 3504 | 0.9 |
| L271P | | 9685 | 5713 | 0.6 |
| V272P | | 2744 | 2037 | 0.7 |
| H261L | Histidine mutation | 15171 | 8716 | 0.6 |

TABLE 2

FACS analysis of untagged RVG mutants

| Mutation | Rationale | MFI at pH 7.4 | MFI at pH 5.8 | Ratio MFI 5.8/7.4 |
|---|---|---|---|---|
| WT | N/A | 205 | 22 | 0.11 |
| H270P | Prevention of F-helix formation | 3991 | 3807 | 0.95 |
| L271P | | 2611 | 1887 | 0.72 |
| V272P | | 2632 | 1689 | 0.64 |
| H261L | | 2083 | 1414 | 0.68 |

Expression was also tested by sandwich ELISA, after extraction of membrane proteins from transfected cells by solubilisation in β-octyl glucoside. In brief, plates were coated with mAb 1112-1. Cell extracts were then applied either at pH 5.8 or pH 7.4. Bound RVG was detected using a chimeric mAb to RVG site III as primary antibody and alkaline phosphatase conjugated anti-human immunoglobulin G-Fc as secondary antibody, followed by incubation with p-nitrophenyl phosphate and measurement of OD405. Quantity of RVG was calculated by interpolation against a standard curve. The results of the ELISA analysis are provided in Table 3.

TABLE 3

ELISA analysis of untapped RVG mutants

| Mutation | Strategy | Relative RVG concentration at pH 7.4 | Relative RVG concentration at pH 5.8 | Ratio MFI 5.8/7.4 |
|---|---|---|---|---|
| WT | N/A | 1056.7 | 338.7 | 0.3 |
| H270P | Prevention of F-helix formation | 3298.1 | 2109.8 | 0.6 |
| L271P | | 3274.9 | 3281.0 | 1.0 |
| V272P | | 2036.3 | 2432.9 | 1.2 |
| H261L | | 1258.9 | 1824.9 | 1.4 |

### Example 2: Generation and Expression of Mutant Herpesvirus gB Proteins

The protein coding sequences of the ectodomains of EBV gB and CMV gB were synthesized, bearing C-terminal hexahistidine and Strep-II tags respectively. The amino acid sequences were as follows:
EBV gB ectodomain (SEQ ID NO: 7);
CMV gB ectodomain with C-terminal Strep-II tag: (SEQ ID NO: 9).

In addition to wild type constructs, constructs were also prepared by substituting a proline for each of residues Q471, 1472, N473, R474 in EBV gB, and Y494, 1495, N496 and R497 in CMV gB (i.e. a total of 8 mutant constructs, each containing a single proline substitution).

The coding sequences were cloned into a transient mammalian expression plasmid and transiently transfected into Expi293 cells using Expifectamine (both from ThermoFisher). Supernatant was collected after four days and proteins purified by affinity chromatography using methods appropriate to the tags. Purity and mass of the resulting proteins were assessed by Coomassie Blue staining of an SDS-PAGE gel. Proteins were subjected to size exclusion chromatography on a Superose 6 column (GE Healthcare). Negative stain microscopy and cryo-electron microscopy were performed using routine techniques.

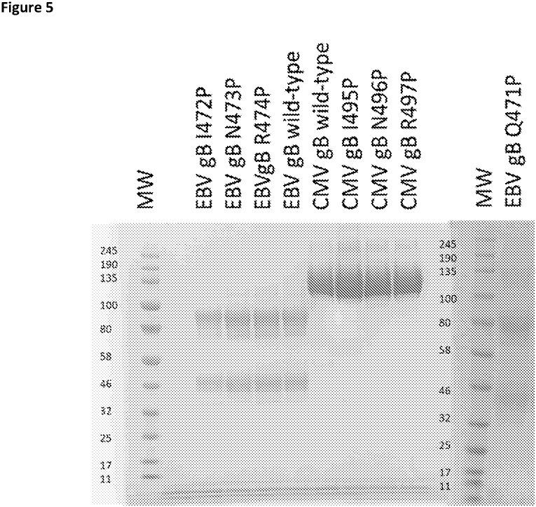

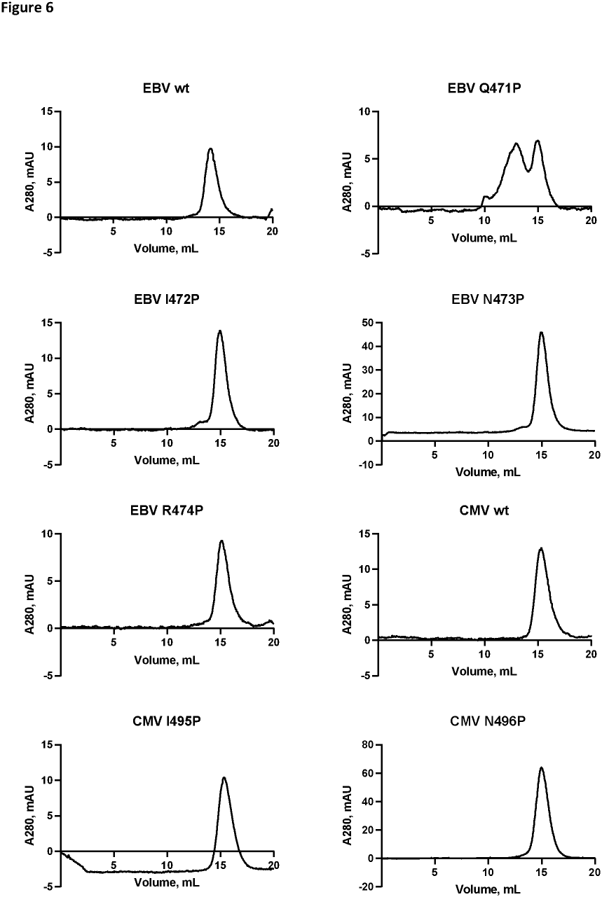

As demonstrated by the Coomassie Blue stained SDS-PAGE gel (FIG. 5) >80% pure preparations of both wild-type and mutant proteins were obtained by single-step affinity chromatography at yields in excess of 10 µg of protein per mL of culture. Size exclusion chromatography illustrated that some but not all constructs migrated as would be expected for trimers of circa 300 kDa (FIG. 6), indicating that these trimeric constructs had not aggregated.

Similar wild-type and mutant constructs are also constructed on the background of full length proteins, including the transmembrane domains (i.e. SEQ ID NO: 6 for EBV gB and SEQ ID NO: 8 for CMV gB), and expressed as described above. The resulting expressed proteins are characterised by cryo-electron microscopy (+/- tomography) on the cell membrane, on membrane protein-enriched extracellular vesicles, or on enveloped virus-like particles such as those based on retroviruses or lentiviruses. Protein trimer spikes are identified, and the length:width aspect ratio of each spike measured. A larger proportion of the mutant protein spikes, as compared to the wildtype protein spikes, have a relatively low aspect ratio, consistent with pre-fusion conformation.”

### Example 3: Assessment of Neutralising Antibody Production in Mice Administered With RVG Mutant Proteins Provided as a Part of a Simian Adenovirus-Vectored Vaccine or as a Protein-Based Vaccine

### Simian Adenovirus-Vectored Vaccine

Mutant variants of rabies glycoprotein according to the invention are cloned into ChAxOX2 adenovirus vector (protein SEQ ID NOs: 11, 12, 13, 14) and viruses produced using CsCl purification method yielding mutant variants of ChAdOX2-RabG vaccine. CD1 strain mice are administered with a range of doses of mutant ChAdOx2-RabG vaccine variants from 1×10³ to 1×10⁸ IU per animal by intramuscular injection. A control group receive the unmodified ChAdOx2-RabG vaccine of the same doses (protein SEQ ID NO: 2).

### Protein-Based Vaccine

Wild-type and mutant variants of RVG protein (SEQ ID NOs: 11, 12, 13, 14) are produced and purified in a soluble form. CD1 strain mice are administered with a range of doses from 10 ng to 10 µg of the RVG mutant proteins per animal by intramuscular injection. A control group receive same amounts of the RVG unmodified protein (SEQ ID NO: 2).

Immunological blood samples are taken at day 0, 7, 14 and 21 with respect to ChAdOx2-RabG/RVG protein administration on day 0. The production of neutralising antibodies against the pre-fusion conformation of RVG is assessed by a variety of immunological assays. No safety/tolerability issues are noted in mice receiving either the mutant ChAdOX2-Rab/RVG protein or control. Mice administered ChAdOX2-Rab_v1.3 carrying H270P mutation or RVG H270P protein (protein SEQ ID NO: 11) show high levels of RVG pre-fusion conformation specific neutralising antibodies by day 7. Additionally, they exhibit a higher longevity of immunological response and/or achieve same level of the response with a lower dose of vaccine/antigen.

### Example 4: Electron Microscopy Characterisation of Protein Mutant Variants of RV, EBV and CMV Glycoproteins

Mutant protein variants of either RVG (SEQ ID NOs: 11-14), EBV gB (SEQ ID NOs:19-22) or CMV gB (SEQ ID NOs:23-26) glycoproteins are prepared in one of the following forms:
1) Soluble form - protein construct comprised of ectodomain and lacking both transmembrane and intracellular regions;
2) Detergent-solubilised form - full-length protein construct comprised of ectodomain, transmembrane and intracellular regions, and isolated in the presence of detergent;
3) Membrane-bound form - full-length protein construct as per 2) bound to the membrane protein-enriched extracellular vesicles (Zeev-Ben-Mordehai et al., (2014), Nat. Commun. 5:3912)
4) VLP-bound form - soluble form of proteins as per 1) bound to the enveloped VLP via Spytag-Spycatcher interactions (Brune & Howarth, Front Immunol (9) 1432, 2018)

Protein isolates (1) and (2) are subjected to negative staining electron transmission microscopy (EM) and cryo-electron transmission microscopy (cryo-EM) in both neutral and acidic conditions. Protein isolates (3) and (4) are subjected to cryo-electron tomography (cryo-ET). Wild-type/unmodified forms of corresponding proteins (RVG SEQ ID NO: 2, EBV gB SEQ ID NO: 6 and CMV gB SEQ ID NO: 8) are used as respective controls and underwent identical procedures. Datasets containing sufficient quantities of particles are recorded and processed using suitable software packages to produce 2D class averages of either single or subtomogram particles. Within 2D class averages, overall shape and particle dimensions are used as criteria to judge occurrence or extent of conformational changes of the WT and mutant protein variants.

### Example 5: Combination of Successful Mutations in RV, EBV and CMV Glycoproteins to Facilitate Pre-Fusion State and Protein Immunogenicity

Based on the experimental data from examples 1-4, the most successful individual mutations (i.e. those providing the highest stabilisation readout in FACS, ELISA and EM/cryo-EM experiments combined with the high protein expression as well as rendering advantageous immunological response) are identified in RV, EBV and CMV glycoproteins. Protein constructs carrying combinations of individual mutations are prepared and tested through the mentioned above series of experiments to determine their superior behaviour as compared to the single-mutation constructs.

The main strategies to combine mutations are the following:
Pairwise combination of proline mutations in different positions;
Pairwise combination of proline mutations with other mutations not mentioned in this patent aiming at other pre-fusion stabilising strategy;
Pairwise combination of proline mutations and mutations that stabilise inter-subunit interactions between monomers in a trimer.

### Example 6: Preparation of Full-Length EBV and CMV Glycoprotein Mutant Proteins

In addition to soluble forms, EBV and CMV wild-type and mutant glycoproteins are produced in a membrane-bound form on the surface of the membrane protein-enriched extracellular vesicles (MPEEVs; Zeev-Ben-Mordehai et al., (2014), Nat. Commun. 5:3912). For this, plasmids encoding full-length EBV gB and CMV gB wild-type and mutant constructs (based on SEQ ID Nos: 6 and 8 and with introduced proline mutations as per SEQ ID Nos: 19-26) are transfected into mammalian cells. The formation of MPEEVs is confirmed using SDS-PAGE/Western blotting analysis and subsequent visualisation with the negative staining EM. Following the established protocol for MPEEVs preparation, isolated samples were subjected to cryo-ET visualisation and analysis as per example 4.

### Example 7: Construction of VLPs Displaying the Antigens

Virus-like particles carrying either RV, EBV or CMV mutant proteins are designed using the following approaches:
1) Lenti-virus based VLP system
2) Spytag-Spycatcher system

### Example 8: Construction of Nucleic-Acid-Based/ Vectored Vaccines Expressing the Antigens

RV, EBV and CMV glycoprotein antigens can be delivered to target organisms using various vaccine delivery platforms such as:
1) DNA-based vaccines;
2) RNA-based vaccines;
3) simian adenovirus-vectored vaccines (see example 3);
4) vaccines vectored by other viruses (e.g. rVSV or MVA).

Such types of vaccines are well established in the field and their preparation methods are published. Vaccines are prepared carrying either single proline or combined (see example 5) mutant variants of RVG, EBV gB and CMV gB proteins and mice are immunised according to established protocols in order to identify the most suitable platform for this study. Advantageous features of the suitable platform might be but not limited to:
A stronger immune response, meaning presence of the higher titers of neutralising antibodies at the same timepoint post-immunisation.
A more durable immune response, meaning longer presence of the sufficient titers of neutralising antibodies at certain timepoint post-immunisation.
Improved immunogenicity expressed as an equivalent immune response using smaller dose of vaccine at the same time point post-immunisation.

### Example 9: Assessment of Neutralising Antibody Production in Mice Administered With EBV gB and CMV gB Mutant Proteins as a Part of Simian Adenovirus-Vectored Vaccine or as a Protein-Based Vaccine

Simian adenovirus-vectored vaccines and protein-based vaccines are generated and tested using EBV gB and CMV gB mutant proteins according to the invention as described for RVG in example 3.

When tested in mice, both mutant EBV gB and CMV gB vaccines resulted in high levels of EBV gB and CMV gB pre-fusion conformation specific neutralising antibodies respectively.

### Example 10: Assessment of Conformational Accuracy and Acid Stability of RVG H270P Mutant By Antibodies Directed to Various Antigenic Epitopes

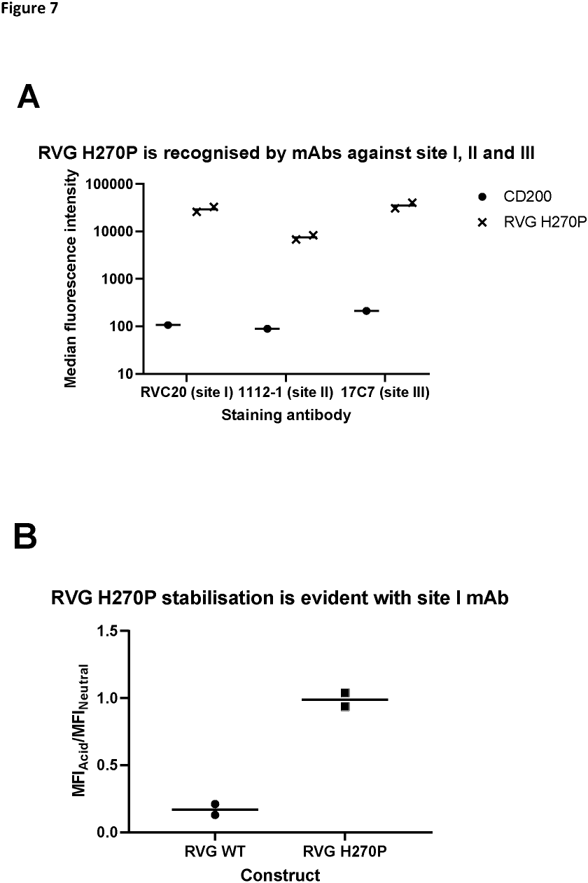

RVG H270P mutant variant has increased stability under acid conditions according to FACS analysis using the antigenic site II-specific antibody, 1112-1 (FIG. 7A). Using two additional antibodies RVC20 (antigenic site I-specific) and 17C7 (antigenic site III-specific) that recognize distinct antigenic epitopes on the RVG surface, the accuracy of the protein conformation under neutral pH and its stability in acidic environments was tested. Antigenic site I is linear and present under neutral conditions (i.e. when the WT RVG is in the pre-fusion conformation), however, it is occluded in the post-fusion conformation. Antigenic site III is conformational. Currently site III-specific antibodies are not considered capable of discriminating between pre-fusion and post-fusion conformations, but binding of site III-specific antibodies serves as a further test that RVG is correctly folded.

Expi293 cells were transfected with DNA encoding either an irrelevant protein (human CD200) or the RVG H270P mutant, then stained with each of the above antibodies at pH 7.4, and then allophycocyanin-(APC) labelled anti-mouse-immunoglobulin. Data was acquired on a FACS Canto cytometer (BD Biosciences) and median fluorescence intensities (MFI) calculated. FIG. 7A shows that the H270P construct is reactive with all three antigenic sites.

In addition, cells were transfected with DNA encoding either wildtype RVG or RVG H270P, then stained with RVC20 (antigenic site I-specific antibody) at either pH 5.8 or pH 7.4, before proceeding with secondary staining (at pH 7.4) and flow cytometry as above. The ratio of MFI under acid vs neutral conditions was calculated. FIG. 7B shows that the H270P mutant retains the RVC20 (site I) epitope under acid conditions, whereas the majority of RVC20 staining of wildtype RVG is lost under acid conditions. This demonstrates that the H270P mutant is stabilised in the pre-fusion conformation, whereas the wildtype RVG is not.

### Discussion

The results obtained illustrate that these designed constructs achieve both enhanced stability of the pre-fusion form, and enhanced cell surface overall expression levels. This stability enhanced the display of an epitope which is known to be a target of protective neutralising antibody. These properties potentially valuable for the production of rabies vaccines: enhanced quality and quantity of expressed antigen will be beneficial for nucleic-acid-based/ vectored vaccines; enhanced manufacturing yield, storage stability and antigen quality (i.e. display of neutralising antibody epitopes) will be beneficial for protein-based and VLP vaccines; achievement of the similar response with less amount of antigen will lead to decreased price per dose of vaccine.

Similarly, stabilised mutants will be of value for herpesvirus vaccines and other viruses with Class III fusion proteins.

1. A stable pre-fusion Class III fusion protein, or an immunogenic fragment thereof, comprising one or more mutations that prevent formation of the central extended helix in the post-fusion conformation trimerisation interface.

2. The protein or immunogenic fragment thereof of claim 1, wherein said one or more mutation that prevents the formation of the central extended helix is a mutation that prevents extension of the pre-fusion conformation central helix into the post-fusion central extended helix.

3. The protein or immunogenic fragment thereof, of claim 1 or 2, wherein said central extended helix in the post-fusion conformation trimerisation interface corresponds to or aligns with:
(a) the extended helix C in rabies virus glycoprotein (RVG);
(b) helix F of the trimerisation domain of vesicular stomatitis virus glycoprotein (VSVG); and/or
(c) helix alpha-C of the trimerisation domain of Epstein-Barr virus glycoprotein B (EBV gB).

4. The protein or immunogenic fragment thereof of any one of the preceding claims, wherein said central extended helix in the post-fusion conformation trimerisation interface is defined as extending, at its N-terminal end, up to the 32^{nd} amino acid residue N-terminal to the conserved cysteine residue of the Class III fusion protein and, at its C-terminal end, up to the 16^{th} amino acid residue C-terminal to the conserved cysteine of the Class III fusion protein; wherein optionally the conserved cysteine residue corresponds to or aligns with:
(a) amino acid residue 283 of the RVG sequence according to SEQ ID NO: 3;
(b) amino acid residue 284 of the VSVG sequence according to SEQ ID NO: 5;
(c) amino acid residue 484 of the EBV gB sequence according to SEQ ID NO: 6;
(d) amino acid residue 507 of the cytomegalovirus glycoprotein B (CMV gB) sequence according to SEQ ID NO: 8; and/or
(e) amino acid residue 529 of the herpes simplex virus glycoprotein B (HSV-1 gB) sequence according to SEQ ID NO: 10.
(f) amino acid residue 526 of the herpes simplex virus glycoprotein B (HSV-2 gB) sequence according to SEQ ID NO: 32.

5. The protein or immunogenic fragment thereof of any one of the preceding claims, wherein said central extended helix of the post-fusion conformation trimerisation interface corresponds to, or aligns with amino acid residues:
(a) 262 to 293 of the RVG sequence of SEQ ID NO: 3;
(b) 263 to 294 of the VSVG sequence of SEQ ID NO: 5;
(c) 458 to 500 of the EBV gB sequence of SEQ ID NO: 6;
(d) 481 to 520 of the CMV gB sequence of SEQ ID NO: 8; and/or
(e) 503 to 545 of the HSV-1 gB sequence of SEQ ID NO: 10.

6. The protein or immunogenic fragment thereof of any one of the preceding claims, wherein the Class III fusion protein is a:
(a) RVG;
(b) VSVG; or
(c) herpesvirus glycoprotein B, wherein optionally said herpesvirus is selected from a cytomegalovirus, Epstein- Barr virus, herpes simplex virus-1 and/or herpes simplex virus-2.

7. The protein or immunogenic fragment thereof of any one of the preceding claims, wherein said one or more mutation is an amino acid substitution.

8. The protein or immunogenic fragment thereof of any one of the preceding claims, wherein said amino acid substitution is a non-conservative amino acid substitution.

9. The protein or immunogenic fragment thereof of any one of the preceding claims, wherein said one or more mutation is:
(a) a helix-disrupting mutation and said amino acid substitution is a substitution by proline; and/or
(b) an amino acid substitution by an amino acid with increased hydrophobicity compared with the amino acid being substituted, wherein said amino acid substitution is independently selected from a substitution by leucine, alanine, isoleucine or valine.

10. The protein or immunogenic fragment thereof of any one of the preceding claims, wherein said one or more mutation is at an amino acid corresponding to, or aligning with, position 270, 271, 272 and/or 273 of the RVG sequence of SEQ ID NO: 3.

11. The protein or immunogenic fragment thereof of any one of the preceding claims, wherein the amino acid corresponding to position:
(a) 270 of the RVG sequence of SEQ ID NO: 3 is substituted by proline;
(b) 271 of the RVG sequence of SEQ ID NO: 3 is substituted by proline;
(c) 272 of the RVG sequence of SEQ ID NO: 3 is substituted by proline; and/or
(d) 273 of the RVG sequence of SEQ ID NO: 3 is substituted by proline.

12. The protein or immunogenic fragment thereof of any one of the preceding claims, wherein the Class III fusion protein is a RVG and wherein said protein comprises one or more amino acid substitutions selected from H270P, L271P, V272P and/or V273P.

13. The protein or immunogenic fragment thereof of any one of the preceding claims, wherein the protein or immunogenic fragment thereof is RVG comprising an amino acid sequence selected from SEQ ID NOs: 11, 12, 13 or 14.

14. The protein or immunogenic fragment thereof of any one of claims 1 to 9, wherein said one or more mutation is at an amino acid corresponding to, or aligning with, position 516, 517, 518 and/or 519 of the HSV gB sequence of SEQ ID NO: 10.

15. The protein or immunogenic fragment thereof of any one of claims 1 to 9 or 14, wherein the amino acid corresponding to position:
(a) 516 of the HSV gB sequence of SEQ ID NO: 10 is substituted by proline;
(b) 517 of the HSV gB sequence of SEQ ID NO: 10 is substituted by proline;
(c) 518 of the HSV gB sequence of SEQ ID NO: 10 is substituted by proline; and/or
(d) 519 of the HSV gB sequence of SEQ ID NO: 10 is substituted by proline.

16. The protein or immunogenic fragment thereof of any one of claims 1 to 9, 14 or 15, wherein the Class III fusion protein is an HSV gB and wherein said protein comprises one or more amino acid substitutions selected from H516P, V517P, N518P and/or D519P.

17. The protein or immunogenic fragment thereof of any one of claims 1 to 9 or 14 to 16, wherein the protein or immunogenic fragment thereof is HSV gB comprising an amino acid sequence selected from SEQ ID NOs: 27, 28, 29 or 30.

18. A stable pre-fusion Class III fusion protein, or an immunogenic fragment thereof, comprising:
(a) one or more non-conservative amino acid substitutions within its pre-fusion conformation central helix; and/or
(b) one or more non-conservative amino acid substitutions within amino acid residues corresponding to or aligning with amino acid residues: (i) 262 to 293 of the RVG sequence of SEQ ID NO: 3; (ii) 263 to 294 of the VSVG sequence of SEQ ID NO: 5; (iii) 458 to 500 of the EBV gB sequence of SEQ ID NO: 6; (iv) 481 to 520 of the CMV gB sequence of SEQ ID NO: 8; and/or (v) 503 to 545 of the HSV-1 gB sequence of SEQ ID NO: 10;
wherein preferably the one or more non-conservative amino acid substitution is substitution by proline.

19. The protein or immunogenic fragment thereof of any one of the preceding claims, which further comprises one or more additional mutations in a region other than the central extended helix of the post-fusion conformation trimerisation interface.

20. The protein or immunogenic fragment thereof of claim 19, wherein said one more additional mutation is a mutation in an amino acid corresponding to or aligning with position:
(a) 261 of the RVG sequence of SEQ ID NO: 3;
(b) 262 of the VSVG sequence of SEQ ID NO: 5;
(c) 462 of the EBV gB sequence of SEQ ID NO: 6;
(d) 485 of the CMV gB sequence of SEQ ID NO: 8; and/or
(e) 507 of the HSV gB sequence of SEQ ID NO: 10;
wherein optionally said amino acid mutation is an amino acid substitution by an amino acid with increased hydrophobicity compared to the amino acid being substituted.

21. The protein or immunogenic fragment thereof of any one of the preceding claims, which induces neutralising antibodies against one or more epitope of the pre-fusion Class III fusion protein.

22. The protein or immunogenic fragment thereof of claim 21, which induces neutralising antibodies against (i) antigenic site I; (ii) antigenic site II; and/or (iii) antigenic site III.

23. The protein or immunogenic fragment thereof of any one of the preceding claims, for use in a vaccine.

24. A polynucleotide molecule encoding a protein as defined in any one of claims 1 to 22.

25. A viral vector, DNA vector and/or RNA vector:
(a) comprising a polynucleotide as defined in claim 24; and/or
(b) encoding a protein or immunogenic fragment thereof as defined in any one of claims 1 to 22.

26. A virus-like particle, comprising a protein as defined in any one of claims 1 to 22.

27. A vaccine composition, comprising a protein as defined in any one of claims 1 to 22, a polynucleotide molecule as defined in claim 24, a viral vector and/or DNA vector and/or RNA vector as defined in claim 25, and/or a virus-like particle as defined in claim 26, and optionally a pharmaceutically acceptable excipient.

28. An antibody, or binding fragment thereof, that specifically binds to the protein or immunogenic fragment thereof as defined in any one of claims 1 to 22.

29. The protein of any one of claims 1 to 22, and/or the vaccine composition of claim 27, and/or the polynucleotide of claim 24, and/or the viral vector and/or DNA vector and/or RNA vector of claim 25 and/or the virus-like particle of claim 26 and/or the antibody of claim 28, for use in a method of immunising a subject against a viral infection.

30. Use of the protein of any one of claims 1 to 22, and/or the vaccine composition of claim 27, and/or the polynucleotide of claim 24, and/or the viral vector and/or DNA vector and/or RNA vector of claim 25, and/or the virus-like particle of claim 26, and/or the antibody of claim 28, in the manufacture of a medicament for the immunisation of a subject against a viral infection.

31. The protein, vaccine composition, polynucleotide, viral vector, DNA vector, RNA vector, virus-like particle and/or antibody for use according to claim 29; or the use of the protein, vaccine composition, polynucleotide, viral vector, DNA vector, RNA vector, virus-like particle and/or antibody according to claim 30; wherein said subject is a mammalian subject, preferably a human subject.

32. The protein, vaccine composition, polynucleotide, viral vector, DNA vector, RNA vector, virus-like particle and/or antibody for use according to claim 29 or 31; or the use of the protein, vaccine composition, polynucleotide, viral vector, DNA vector, RNA vector, virus-like particle and/or antibody according to claim 30 or 31; wherein said viral infection is a rhabdovirus infection or a herpesvirus infection.

33. Use of the protein of any one of claims 1 to 22 for the generation of an antibody, or binding fragment thereof, specific to an epitope of a pre-fusion conformation Class III fusion protein.


SEQUENCE LISTING (32 sequences)

SEQ ID NO: 1 - protein, 524 aa, Rabies virus
MVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGL

SEQ ID NO: 2 - protein, 524 aa, Artificial Sequence
  Chimeric RVG
MVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSPVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESHKSGGETRL

SEQ ID NO: 3 - protein, 505 aa, Artificial Sequence
  Chimeric RVG
KFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSPVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESHKSGGETRL

SEQ ID NO: 4 - protein, 511 aa, Vesicular stomatitis virus
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 5 - protein, 495 aa, Vesicular stomatitis virus
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 6 - protein, 857 aa, Human herpesvirus 4
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 7 - protein, 714 aa, Artificial Sequence
  Epstein-Barr virus expression construct
MDAMKRGLCCVLLLCGAVFVSPSASQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGGSGSGHHHHHHGLNDIFEAQKIEWHE

SEQ ID NO: 8 - protein, 907 aa, Artificial Sequence
  Human cytomegalovirus gB
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 9 - protein, 709 aa, Artificial Sequence
  Cytomegalovirus gB expression construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEGSAWSHPQFEK

SEQ ID NO: 10 - protein, 904 aa, Artificial Sequence
  Herpes Simplex Virus 1 gB
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 11 - protein, 505 aa, Artificial Sequence
  RVG comprising the H270P mutation
KFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSPVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEPLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESHKSGGETRL

SEQ ID NO: 12 - protein, 505 aa, Artificial Sequence
  RVG comprising the L271P mutation
KFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSPVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHPVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESHKSGGETRL

SEQ ID NO: 13 - protein, 505 aa, Artificial Sequence
  RVG comprising the V272P mutation
KFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSPVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLPVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESHKSGGETRL

SEQ ID NO: 14 - protein, 505 aa, Artificial Sequence
  RVG comprising the V273P mutation
KFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSPVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVPEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESHKSGGETRL

SEQ ID NO: 15 - protein, 495 aa, Artificial Sequence
  VSVG comprising the L271P mutation
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSPIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 16 - protein, 495 aa, Artificial Sequence
  VSVG comprising the I272P mutation
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLPQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 17 - protein, 495 aa, Artificial Sequence
  VSVG comprising the Q273P mutation
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIPDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - protein, 495 aa, Artificial Sequence
  VSVG comprising the D274P mutation
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQPVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 19 - protein, 714 aa, Artificial Sequence
  EBV gB comprising the Q471P mutation
MDAMKRGLCCVLLLCGAVFVSPSASQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRPINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGGSGSGHHHHHHGLNDIFEAQKIEWHE

SEQ ID NO: 20 - protein, 714 aa, Artificial Sequence
  EBV gB comprising the I472P mutation
MDAMKRGLCCVLLLCGAVFVSPSASQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQPNRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGGSGSGHHHHHHGLNDIFEAQKIEWHE

SEQ ID NO: 21 - protein, 714 aa, Artificial Sequence
  EBV gB comprising the N473P mutation
MDAMKRGLCCVLLLCGAVFVSPSASQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQIPRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGGSGSGHHHHHHGLNDIFEAQKIEWHE

SEQ ID NO: 22 - protein, 714 aa, Artificial Sequence
  EBV gB comprising the R474P mutation
MDAMKRGLCCVLLLCGAVFVSPSASQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINPMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGGSGSGHHHHHHGLNDIFEAQKIEWHE

SEQ ID NO: 23 - protein, 589 aa, Artificial Sequence
  CMV gB comprising the Y494P mutation
LDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGPINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEGSAWSHPQFEK

SEQ ID NO: 24 - protein, 709 aa, Artificial Sequence
  CMV gB comprising the I495P mutation
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYPNRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEGSAWSHPQFEK

SEQ ID NO: 25 - protein, 709 aa, Artificial Sequence
  CMV gB comprising the N496P mutation
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYIPRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEGSAWSHPQFEK

SEQ ID NO: 26 - protein, 709 aa, Artificial Sequence
  CMV gB comprising the R497P mutation
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINPALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEGSAWSHPQFEK

SEQ ID NO: 27 - protein, 904 aa, Artificial Sequence
  HSV-1 gB comprising the H516P mutation
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRPVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 28 - protein, 904 aa, Artificial Sequence
  HSV-1 gB comprising the V517P mutation
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHPNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 29 - protein, 904 aa, Artificial Sequence
  HSV-1 gB comprising the N518P mutation
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVPDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 30 - protein, 904 aa, Artificial Sequence
  HSV-1 gB comprising the D519P mutation
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNPMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 31 - protein, 524 aa, Rabies virus
MVPQALLFVPLLVFPLCFGKFPIYTIPDKLGPWSPIDIHHLSCPNNLVVEDEGCTNLSGFSYMELKVGYILAIKVNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVSMQTSNETKWCPPDKLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 32 - protein, 904 aa, Artificial Sequence
  Herpes Simplex Virus-2 gB
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL